SEQUENCE LISTING (68 sequences)

SEQ ID NO: 1 - DNA, 40 bp, Homo sapiens
  source 1..40: mol_type = genomic DNA
tgtgcagcactctaactttggaaatgagaaattaaccttt

SEQ ID NO: 2 - DNA, 40 bp, Homo sapiens
  source 1..40: mol_type = genomic DNA
tgtgccagtgaactcaaattccgggtatgcactcaacttc

SEQ ID NO: 3 - DNA, 40 bp, Homo sapiens
  source 1..40: mol_type = genomic DNA
tgtgcccagaactaactttggaaatgagaaattaaccttt

SEQ ID NO: 4 - DNA, 45 bp, Homo sapiens
  source 1..45: mol_type = genomic DNA
tgtgcttcagaggcgttttctggtggctacaataagctgattttt

SEQ ID NO: 5 - DNA, 35 bp, Homo sapiens
  source 1..35: mol_type = genomic DNA
gggacaggggggggagagacccagtacttcgggcc

SEQ ID NO: 6 - DNA, 28 bp, Homo sapiens
  source 1..28: mol_type = genomic DNA
gggactcctacgagcagtacgtcgggcc

SEQ ID NO: 7 - DNA, 33 bp, Homo sapiens
  source 1..33: mol_type = genomic DNA
gggactgtccgcgcacgagcagtacttcgggcc

SEQ ID NO: 8 - DNA, 54 bp, Homo sapiens
  source 1..54: mol_type = genomic DNA
gggacagcaaatgtcacccgaagttaagatcacagatacgcagtattttggccc

SEQ ID NO: 9 - DNA, 44 bp, Homo sapiens
  source 1..44: mol_type = genomic DNA
tgtgccagcagctagggggagggaacaccggggagctgtttttt

SEQ ID NO: 10 - DNA, 44 bp, Homo sapiens
  source 1..44: mol_type = genomic DNA
tgtgccagcagctgtcgagcggggtcaccggggagctgtttttt

SEQ ID NO: 11 - DNA, 45 bp, Homo sapiens
  source 1..45: mol_type = genomic DNA
tgtgccagcagctccaagggggatgacgatcagccccagcatttt

SEQ ID NO: 12 - DNA, 34 bp, Homo sapiens
  source 1..34: mol_type = genomic DNA
tgtgccagcagctcgggactagagcacgttcttc

SEQ ID NO: 13 - DNA, 32 bp, Homo sapiens
  source 1..32: mol_type = genomic DNA
tgtgccttgtgggaggtgggaggaaactcttt

SEQ ID NO: 14 - DNA, 42 bp, Homo sapiens
  source 1..42: mol_type = genomic DNA
tgtgcctgctggcccctcactcttcagcataagaaactcttt

SEQ ID NO: 15 - DNA, 38 bp, Homo sapiens
  source 1..38: mol_type = genomic DNA
tgtgccacctgggacgggcggaatataagaaactcttt

SEQ ID NO: 16 - DNA, 35 bp, Homo sapiens
  source 1..35: mol_type = genomic DNA
tgtgctgcgtgggattctcttaatcgaaactcttt

SEQ ID NO: 17 - DNA, 42 bp, Homo sapiens
  source 1..42: mol_type = genomic DNA
gtcttactactgtgcctgtgacacctcactgggggatacgca

SEQ ID NO: 18 - DNA, 37 bp, Homo sapiens
  source 1..37: mol_type = genomic DNA
gtcttactactgtgcctgtgacccccagggatacgca

SEQ ID NO: 19 - DNA, 47 bp, Homo sapiens
  source 1..47: mol_type = genomic DNA
gtcttactactgtgcctgtgaccaccctttgtactgggggatacgca

SEQ ID NO: 20 - DNA, 43 bp, Homo sapiens
  source 1..43: mol_type = genomic DNA
gtcttactactgtgcctgtgacaccgggagaaggggatacgca

SEQ ID NO: 21 - DNA, 40 bp, Homo sapiens
  source 1..40: mol_type = genomic DNA
atgtgtttcattgtgccttccccctccggggggatacgca

SEQ ID NO: 22 - DNA, 37 bp, Homo sapiens
  source 1..37: mol_type = genomic DNA
atgtgtttcattgtgccttcccggggggggatacgca

SEQ ID NO: 23 - DNA, 44 bp, Homo sapiens
  source 1..44: mol_type = genomic DNA
atgtgtttcattgtgccttcccggcccgtactgggggatacgca

SEQ ID NO: 24 - DNA, 35 bp, Homo sapiens
  source 1..35: mol_type = genomic DNA
atgtgtttcattgtgcccccttcgggggatacgca

SEQ ID NO: 25 - DNA, 46 bp, Homo sapiens
  source 1..46: mol_type = genomic DNA
catcctgactgggggcccccttccatacaccgataaactcatcttt

SEQ ID NO: 26 - DNA, 54 bp, Homo sapiens
  source 1..54: mol_type = genomic DNA
cattgtgccttccccccgggtctgggggatccgcccaccgataaactcatcttt

SEQ ID NO: 27 - DNA, 48 bp, Homo sapiens
  source 1..48: mol_type = genomic DNA
cattgtgccttcccaactgggggatactggaccgataaactcatcttt

SEQ ID NO: 28 - DNA, 38 bp, Homo sapiens
  source 1..38: mol_type = genomic DNA
cattgtgccttgcgggggggatacataaactcatcttt

SEQ ID NO: 29 - DNA, 58 bp, Homo sapiens
  source 1..58: mol_type = genomic DNA
tgtgcctgtggcggattactgggggatcccaaatactggaggcttcaaaactatcttt

SEQ ID NO: 30 - DNA, 50 bp, Homo sapiens
  source 1..50: mol_type = genomic DNA
tgtgcctgtcagtacccggggtccaccgggttaataggaaactgacattt

SEQ ID NO: 31 - DNA, 44 bp, Homo sapiens
  source 1..44: mol_type = genomic DNA
tgtgccgaacgtgctctaactttggaaatgagaaattaaccttt

SEQ ID NO: 32 - DNA, 56 bp, Homo sapiens
  source 1..56: mol_type = genomic DNA
tgtgcctgtgacaccccgaggggggataaataacaggaaacacacctcttgtcttt

SEQ ID NO: 33 - DNA, 77 bp, Homo sapiens
  source 1..77: mol_type = genomic DNA
aggatattgtagtagtaccagctgccgtcggctacggtgactacgtccccggctgaatacttccagcactggggcca

SEQ ID NO: 34 - DNA, 46 bp, Homo sapiens
  source 1..46: mol_type = genomic DNA
atataacgatttttggagttgaggatgcttttgatatatggggcca

SEQ ID NO: 35 - DNA, 31 bp, Homo sapiens
  source 1..31: mol_type = genomic DNA
tgactacgttggcttttgatatctggggcca

SEQ ID NO: 36 - DNA, 69 bp, Homo sapiens
  source 1..69: mol_type = genomic DNA
gtattactatgatagtagtggttatttaaaatattactactactactacggtatggacgtctggggcca

SEQ ID NO: 37 - DNA, 68 bp, Homo sapiens
  source 1..68: mol_type = genomic DNA
tgtgcgagaagcttaagggcctattactatgatagtagtggttattactacgacactttgactactgg

SEQ ID NO: 38 - DNA, 35 bp, Homo sapiens
  source 1..35: mol_type = genomic DNA
tgtgcgagagtacccagctacctgatgactactgg

SEQ ID NO: 39 - DNA, 48 bp, Homo sapiens
  source 1..48: mol_type = genomic DNA
tgtgcgagctctccgtatagcagcagctggttttcttggtactactgg

SEQ ID NO: 40 - DNA, 50 bp, Homo sapiens
  source 1..50: mol_type = genomic DNA
tgtgctatgggcctttctatagcagctcgtacaactggttcgacccctgg

SEQ ID NO: 41 - DNA, 31 bp, Homo sapiens
  source 1..31: mol_type = genomic DNA
tgtcaacagtatgatcgatgcgctcactttc

SEQ ID NO: 42 - DNA, 33 bp, Homo sapiens
  source 1..33: mol_type = genomic DNA
tgtcaacagcttaatagttacccgatcaccttc

SEQ ID NO: 43 - DNA, 35 bp, Homo sapiens
  source 1..35: mol_type = genomic DNA
ggtcaacggacttacaatgcccctccgggggttct

SEQ ID NO: 44 - DNA, 28 bp, Homo sapiens
  source 1..28: mol_type = genomic DNA
tgtcaacagtatgataatctccggcttc

SEQ ID NO: 45 - DNA, 42 bp, Homo sapiens
  source 1..42: mol_type = genomic DNA
ccagctttcctgataggccctagtggcagcccagggcgactc

SEQ ID NO: 46 - DNA, 40 bp, Homo sapiens
  source 1..40: mol_type = genomic DNA
ccagctttcccggagccctagtggcagcccagggcgactc

SEQ ID NO: 47 - DNA, 40 bp, Homo sapiens
  source 1..40: mol_type = genomic DNA
ccagctttcctgataccctagtggcagcccagggcgactc

SEQ ID NO: 48 - DNA, 39 bp, Homo sapiens
  source 1..39: mol_type = genomic DNA
ccagctttcctgagccctagtggcagcccagggcgactc

SEQ ID NO: 49 - DNA, 31 bp, Homo sapiens
  source 1..31: mol_type = genomic DNA
agtcccggggagtggcagcccagggcgactc

SEQ ID NO: 50 - DNA, 31 bp, Homo sapiens
  source 1..31: mol_type = genomic DNA
atgcccgccgggtggcagcccagggcgactc

SEQ ID NO: 51 - DNA, 36 bp, Homo sapiens
  source 1..36: mol_type = genomic DNA
actggccccggccctagtggcagcccagggcgactc

SEQ ID NO: 52 - DNA, 23 bp, Homo sapiens
  source 1..23: mol_type = genomic DNA
atctcccgacgacggggcgactc

SEQ ID NO: 53 - DNA, 33 bp, Homo sapiens
  source 1..33: mol_type = genomic DNA
tgcagctcatatgcaggcagcaactgggtgttc

SEQ ID NO: 54 - DNA, 39 bp, Homo sapiens
  source 1..39: mol_type = genomic DNA
tgtcaatcagcagacagcagtggtacttatgtggtattc

SEQ ID NO: 55 - DNA, 36 bp, Homo sapiens
  source 1..36: mol_type = genomic DNA
tgcagctcatatgcaggcagcctttatgtggtattc

SEQ ID NO: 56 - DNA, 34 bp, Homo sapiens
  source 1..34: mol_type = genomic DNA
tgtcaggcgtgggacagcagcactgcatgtcttc

SEQ ID NO: 57 - DNA, 38 bp, Homo sapiens
  source 1..38: mol_type = genomic DNA
cttctacatctgcagtgctagagggactagcgggaggg

SEQ ID NO: 58 - DNA, 40 bp, Homo sapiens
  source 1..40: mol_type = genomic DNA
tgtgtatttctgtgccagcagccaagattgggcggggggg

SEQ ID NO: 59 - DNA, 33 bp, Homo sapiens
  source 1..33: mol_type = genomic DNA
cttctacatctgcagtgctcttaagcggggggg

SEQ ID NO: 60 - DNA, 45 bp, Homo sapiens
  source 1..45: mol_type = genomic DNA
tgtgtatttctgtgccagcagccaagatcccgcctggcggggggg

SEQ ID NO: 61 - DNA, 23 bp, Homo sapiens
  source 1..23: mol_type = genomic DNA
gggacaggggactagcgggaggg

SEQ ID NO: 62 - DNA, 27 bp, Homo sapiens
  source 1..27: mol_type = genomic DNA
gggacaggggggtagctagcgggaggg

SEQ ID NO: 63 - DNA, 27 bp, Homo sapiens
  source 1..27: mol_type = genomic DNA
gggacagggggcgcccctgttagaggg

SEQ ID NO: 64 - DNA, 31 bp, Homo sapiens
  source 1..31: mol_type = genomic DNA
gggacagttaaggctagggctagcggggggg

SEQ ID NO: 65 - DNA, 46 bp, Homo sapiens
  source 1..46: mol_type = genomic DNA
tgtgcgagagcataccctgaatcttgggtatagcagtggctggtac

SEQ ID NO: 66 - DNA, 24 bp, Homo sapiens
  source 1..24: mol_type = genomic DNA
tgtgcacaccccagtggctggtac

SEQ ID NO: 67 - DNA, 28 bp, Homo sapiens
  source 1..28: mol_type = genomic DNA
tgtgcacaccgatgactacagtaactac

SEQ ID NO: 68 - DNA, 27 bp, Homo sapiens
  source 1..27: mol_type = genomic DNA
tgtgcgagtgtgagtaacggtgactac

(19) United States
(12) Patent Application Publication
(10) Pub. No.: US 2025/0297318 A1
(43) Pub. Date: Sep. 25, 2025

(54) DNA-BASED ADAPTOME PROFILING FOR MINIMAL RESIDUAL DISEASE QUANTIFICATION IN LYMPHOID MALIGNANCIES

(71) Applicant: MILABORATORIES INC., San Francisco, CA (US)

(72) Inventors: Alexander KOMKOV, San Francisco, CA (US); Michael MASCHAN, San Francisco, CA (US); Ilgar MAMEDOV, San Francisco, CA (US)

(21) Appl. No.: 18/615,441

(22) Filed: Mar. 25, 2024

(51) Int. Cl. C12Q 1/6886; C12N 15/10; C12Q 1/686; C12Q 1/6869

(52) U.S. Cl. CPC C12Q 1/6886; C12N 15/1093; C12Q 1/686; C12Q 1/6869; C12Q 2600/156

(57) ABSTRACT

The present disclosure relates to target sequencing of T and B cell receptor gene rearrangements at the DNA level and using this technology to detect and quantify lymphoid malignant T or B cells (minimal residual disease, MRD) during and after therapy.

### FIELD

The present disclosure is directed to methods for determining the presence and quantification of minimal residual disease (MRD) level in patients. The methods incorporate multiplex amplification and sequencing of T and B cell receptor gene rearrangements, including incomplete rearrangements, to detect lymphoid malignant T or B cells related to MRD during and after therapy.

### SEQUENCE LISTING

This application incorporates by reference a Sequence Listing submitted with the application as XML file entitled 234-0005US1 SL, created on Mar. 25, 2024, and having a size of 64 kilobytes.

### BACKGROUND

Minimal residual disease (MRD) refers to a small amount of cancer cells remaining in a patient following treatment that cannot be detected using standard cancer scans or laboratory tests. As MRD is likely to lead to relapse, its prompt detection and determining the MRD level (quantification, concentration of leukemic cells) is important for relapse prediction and therapy outcome evaluation.

Immunological based testing for surface proteins on white blood cells can be used for MRD testing in leukemias and lymphomas. However, these tests have a limit of detection of around 1 in 10,000 cells and can only be used in detecting leukemias with a stable cell immunophenotype.

Some current MRD tests are based on detecting a DNA sequence specific to the presence of the cancer. DNA markers tested for include chromosomal translocations, microsatellites, point mutations, immunoglobulin or T cell receptor sequences. Additional sequencing tests are RNA based, but these tests are essentially limited to detecting chromosomal translocations.

### SUMMARY

In aspects, the disclosure provides a method of determining the presence and quantification of minimal residual disease (MRD) in patients with hematological malignancies, the method comprising: (a) detecting rearrangements of T cell receptor genes and B cell receptor genes characteristic for malignant clones in an initial sample from a patient using: a multiplex polymerase chain reaction (PCR) with isolated genomic DNA wherein the PCR reaction comprises a library of oligonucleotides amplifying one or more of the following: TRα/β/γ/δ, IgH/K/λ, DJ, DD, VD, and kappa-deleting element (KDE) rearrangements in a single multiplex mixture of oligonucleotides; high-throughput sequencing of the obtained PCR products; extracting complete and incomplete rearrangements by mapping potential rearrangements to a library comprising V, D, and J gene segments, kappa deletion element (KDE), and IGKC intron sequences, wherein the extraction is performed using a semi-global alignment algorithm to identify flanking sequences, followed by a clustering algorithm correcting for PCR and sequencing errors to assemble complete and/or incomplete rearrangement clonotypes; determining precise clonotype sizes from hematological malignancy-related repertoire; and malignancy-related clonotype detection; and (b) performing follow-up monitoring of MRD in at least 4 independent PCR reactions with isolated and quantified genomic DNA obtained at a follow-up time point; wherein the concentration of a malignant clone in the follow-up time point is determined based on the proportion of independent PCR reactions where the corresponding rearrangement characteristic for the malignant clone is observed.

In aspects of the method, the library of oligonucleotides for multiplex PCR is designed to decrease primer dimer formation and minimize nucleotide diversity.

In aspects of the method, the library of oligonucleotides is designed by a method comprising:
a. dividing target genomic DNA regions into k-mers of a certain length;
b. clustering the target genomic DNA regions using Hamming distances not exceeding 4, not exceeding 5, or not exceeding 6, or not exceeding 7 nucleotides; and
c. identifying clusters of highly similar k-mers according to the selected Hamming distance.

In aspects of the method, the k-mers have a length of at least 16 bases.

In aspects of the method, from the clusters identified, tables of 5-mers in the reverse orientation and 5-mers at the end of 3′ end of the nucleotide sequences in the forward orientation are made. In aspects, the 5-mers tables are intersected pairwise, and the primer set with the lowest 5-mers overlap is selected for further analysis.

In aspects of the method, to equilibrate the annealing temperature of the primers, up to 14 additional target-site-related nucleotides are added to the 5′-end of 18-mers.

In aspects of the method, the intersection procedure using 5-mers is repeated.

In aspects of the method, at least 2 working sets of forward primers and at least 2 working sets of reverse primers are generated.

In aspects of the method, the reverse primer set comprises primers for J-genes, downstream introns for D-genes and KDE. In aspects, the forward primer set comprises primers for V-genes, upstream introns for D-genes and C-intron for IgK. In aspects, the multiplex primer sets generated can be used for separate amplification of complete VJ and/or VDJ rearrangements at TRα, TRβ, TRγ, TRδ, IgH, IgK, IgL loci; partial DJ rearrangements at TRβ, TRδ, IgH loci; VD and DD rearrangements at TRδ and TRβ loci; chimeric TRDV-TRAJ rearrangements; or Kappa deletion rearrangements.

In aspects of the method, dual indexing of each aliquot amplicon and introduction of adapters for sequencing are performed using “step-out” PCR.

In aspects of the method, the average lengths of target amplicons in the 4 multiplex primer sets are: set 1—about 120 bp, set 2—about 160 bp, set 3—about 240 bp, and set 4—about 280 bp.

In aspects of the method, amplicons obtained with a set of primers cannot be a matrix for PCR with the subsequent set of primers.

In aspects of the method, the concentrations of primers in the multiplex mixture are optimized by analyzing the frequency ratios of non-functional rearrangements of T- and B-cell receptor genes.

In aspects of the method, an overamplification rates (OAR) is determined for at least one rearrangement, and the concentration of the primer detecting the rearrangement is adjusted to correct for the amplification bias including overamplification and underamplification). In aspects, an overamplification rates (OAR) is determined for at least one rearrangement to computationally correct for the amplification bias including overamplification and underamplification.

In aspects of the method, a ratio of weighted frequencies (proportion of reads) to unweighted frequencies (proportion of clonotypes) for V, D and J genes is determined. In aspects, if the ratio is a substantial deviation from 1, it is indicative of quantitative bias occurring during amplification.

In aspects of the method, the concentrations of primers in the multiplex mixture is selected to minimize the deviation from the expected value of one for the ratio of weighted frequencies to unweighted frequencies.

In aspects of the method, the MRD detection sensitivity depends exclusively on the available input DNA and sequencing coverage.

In aspects of the method, TRα/β/γ/δ, IgH/K/λ, DJ, DD, VD, and kappa-deleting element (KDE) rearrangements are detected in a single multiplex mixture of oligonucleotides.

### BRIEF DESCRIPTION OF THE DRAWINGS

FIG. 1 is a schematic showing positions of designed multiplex primer sets relative to each other in rearranged TCR and BCR genes as described in Example 1.

FIG. 2 is a plot of sample intensity vs. size for a capillary electrophoresis separation of ready-for-sequencing amplicons (target amplicon+sequencing adapters) as described in Example 1. Ready-for-sequencing amplicons were obtained via PCR using four combinations of designed multiplex primer sets: set2+set3, set2+set4, set1+set3, set1+set4.

FIGS. 3A-M are plots of the frequency of rearrangements detected using the 7GENES multiplex mix as described in Example 2. The average usage of each gene segment (black columns) and proportion of samples in which the corresponding gene segment was identified (gray columns). Next-generation sequencing (NGS) data was obtained with the 7GENES multiplex mix from 190 human PBMC samples and are shown for IGHV, IGHD, IGKV, IGLV, TRBV and TRBD, TRGV, TRDV, TRDD, IGHJ, IGKJ, IGLJ, TRAJ, TRBJ, and TRDJ gene segments as indicated in FIGS. 3A-3M.

FIG. 4 is a plot showing the correlation between expected and observed concentration of leukemic cells in samples with added spike-in as described in Example 3. r—Pearson's correlation coefficient, R2—squared r. Black dots represent the mean value for MRD calculated using three different markers, whiskers show standard deviation.

FIG. 5 shows a plot of the percentage of samples [log10] having the MRD value indicated, in percent, as described in Example 6.

FIGS. 6A and 6B show plots of TRAV (6A) and TRAJ (6B) overamplification rates before (black) and after (grey) optimization of PCR primer concentrations as described in Example 7.

FIGS. 7A and 7B show plots of TRAV (7A) and TRAJ (7B) overamplification rates before (black) and after (grey) residual amplification bias correction using a computational approach as described in Example 7.

### DETAILED DESCRIPTION

The present disclosure provides improved methods for deterring minimal residual disease (MRD) using adaptome profiling. In aspects, the methods are used for detecting MRD in patients with hematological malignancies, e.g., blood cancers.

In this specification and the appended claims, the singular forms “a,” “an” and “the” include plural reference unless context clearly dictates otherwise. Unless defined otherwise, all technical and scientific terms used herein have the same meaning as commonly understood to a person of ordinary skill in the art in the field.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. Unless defined otherwise, all technical and scientific terms used herein have the same meaning as commonly understood to one of ordinary skill in the art to which this disclosure belongs. Although any methods, devices and materials similar or equivalent to those described herein can be used in the practice or testing of the disclosure, the preferred methods, devices and materials are now described.

All publications and patents cited in this specification are herein incorporated by reference as if each individual publication or patent were specifically and individually indicated to be incorporated by reference. The citation of any publication is for its disclosure prior to the filing date and should not be construed as an admission that the present disclosure is not entitled to antedate such publication by virtue of prior disclosure.

It should be understood that the materials and methods proposed herein are not limited to particular compositions or process steps, as they may vary. It is pointed out that, as used in this specification and the appended claims, singular forms include the corresponding plural forms, unless the context clearly dictates otherwise.

The term “antigen-recognizing receptor” as used herein is equivalent to “immune receptor” and refers to T cell receptor (TCR) and B cell receptor (BCR).

The following abbreviations may be used when referring to genes for which rearrangements are detected: immunoglobulin heavy chain variable gene segment (IGHV); immunoglobulin heavy chain diversity gene segment (IGHD); immunoglobulin kappa variable gene segment (IGKV); immunoglobulin lambda variable gene segment (IGLV); T-cell receptor beta chain variable gene segment (TRBV); T-cell receptor beta chain diversity gene segment (TRBD); T-cell receptor alpha chain variable gene segment (TRAV); T-cell receptor gamma chain variable gene segment (TRGV); T-cell receptor delta chain variable gene segment (TRDV); T-cell receptor delta chain diversity gene segment (TRDD); immunoglobulin heavy chain joining gene segment (IGHJ); immunoglobulin kappa chain joining gene segment (IGKJ); immunoglobulin lambda chain joining gene segment (IGLJ); T-cell receptor alpha chain joining gene segment (TRAJ); T-cell receptor beta chain joining gene segment (TRBJ); T-cell receptor gamma chain joining gene segment (TRGJ); and T-cell receptor delta chain joining gene segment (TRDJ).

The term “immune receptor repertoires” as used herein refers to TCR and/or BCR repertoires.

The term “BCR” as used herein refers to B cell receptors, antibodies, or immunoglobulins.

As used herein, the term “CDR” refers to complementarity determining regions—the regions of the antigen-recognizing receptors located in the variable domains of the polypeptide chains of TCRs or BCRs that largely determine the specificity of immune receptor. Variable segment of each chain of the antigen-recognizing receptor consists of three CDRs and four framework regions (FR) located from the amino-to carboxyl terminus in the following order: FR1, CDR1, FR2, CDR2, FR3, CDR3, FR4.

The terms “patient” and “individual” refer to a vertebrate, in particular to a representative of mammalian species, and include, but are not limited to, pets, sports animals, primates, including humans. In specific aspects, the patients are humans.

As used herein, the term “cohort of individuals” refers to a group of patients of the same species suffering from the same disease, or individuals undergoing the same type of therapy or vaccination. In specific aspects, the term refers to a group of people.

As used herein, the term “experimental data” refers to the sequencing data of biological samples obtained from individuals belonging to the analyzed cohort.

As used herein, the term “biological sample” refers to a sample of peripheral blood, a sample of cells or a tissue sample, for example, a biopsy or puncture material taken from a patient, and to cell cultures derived from patient cells.

In some aspects, the cells of interest are cultured and differentiated in vitro before use.

The term “gene segments” is used to describe the segments of the genes that are involved in the generation of TCRs and BCRs.

The term “nucleotide sequences” refers to nucleic acid sequences, including DNA, such as genomic DNA or a cDNA molecule, or RNA. As used herein, the term “cDNA” refers to nucleic acids that have sequence elements complementary to the native mature mRNA species, where the sequence elements are exons and the 5′ and 3′ non-coding regions.

As used herein, the term “sample preparation” refers to all manipulations to which nucleic acids are subjected after purification and before sequencing.

The length of a nucleotide sequence is the number of nucleotides it consists of. The “Hamming distance” is a measure of similarity for two nucleotide sequences with the same length. It is equal to the number of nucleotide substitutions required to change a given nucleotide sequence to target one.

For the purposes of the present disclosure, the length of the compared sequences may be the same as the lengths of the CDR3 regions of the TCR or BCR chains being analyzed, or may be extended to the portion of or to the whole sequences of V and/or J gene segments, or may represent a portion of CDR3.

Sequence comparison is performed by methods known to those skilled in the art. For example, the algorithm described in [Altschul et al. J. Mol. Biol., 215, pp. 403-10 (1990)] may be employed for sequence comparison. As another example, to determine the level of identity and similarity between nucleotide or amino acid sequences, the Blast software package provided by National Center for Biotechnology Information (ncbi.nlm.nih.gov/blast).

Reference to the nucleotide sequence coding amino acid sequence means that this amino acid sequence is produced from the nucleotide sequence during translation of mRNA. As is obvious to any person skilled in the art, the term also includes degenerate nucleotide sequences encoding the same amino acid sequence.

Immunoglobulins consist of heavy chain immunoglobulins (IgH) with constant regions (α, δ, ε, γ, or μ) or light chain immunoglobulins (IgK or IgL) with constant regions λ or κ. An antibody has two identical light chains and two identical heavy chains. Each chain is composed of a constant (C) and a variable region. For the heavy chain, the variable region is composed of a variable (V), diversity (D), and joining (J) segments. Several distinct sequences coding for each type of these segments are present in the genome. A specific VDJ recombination event occurs during the development of a B-cell, marking that cell to generate a specific heavy chain. Diversity in the light chain is generated in a similar fashion except that there is no D region so there is only VJ recombination. Between the joined segments, random addition or deletion of one or several nucleotides may occur, further increasing the diversity of heavy and light chains generated by naïve B-cells. The possible diversity of the antibodies generated by B-cells is then the product of the different heavy and light chains. The variable regions of the heavy and light chains contribute to form the antigen recognition (or binding) region or site. Added to this diversity is a process of somatic hypermutation of VDJ regions, which can occur after a specific response is mounted against some epitope, in germinal centers of lymph nodes and other secondary lymphoid tissues.

A “T cell receptor”, also referred to as “TCR”, is a heterodimeric protein complex located on the T lymphocyte surface. This receptor is present only on T lymphocytes. The main function of TCR is the specific recognition of processed antigens represented by molecules of the main histocompatibility complex (MHC, or HLA). Some TCRs may recognize “nonclassical” MHC molecules, such as CD1d, CD1c, MR1, and others, that may present lipid or other low molecular weight molecules.

A human TCR consists of two subunits, TCRα (TCR alpha) and TCRβ (TCR beta), or TCRγ (TCR gamma) and TCRδ (TCR delta) chains, interconnected by a disulfide bond and presented on the T cell membrane. Each of the TCR chains has an N-terminal variable (V) domain, a junction domain (J) and a constant (C) domain coupled to a transmembrane domain that fixes the receptor in the plasma membrane of the T lymphocyte. The TCR usually and mostly interacts with the MHC antigen complex with six complementarity determining regions (CDRs): three alpha chain and three beta chain regions. In some cases, TCRα chain interaction with antigen dominates. More usually, TCRβ chain interaction with antigen dominates.

In addition to complete V-J and V-D-J rearrangements, the genomes of T and B cells can contain partial (incomplete) D-J, V-D, and D-D rearrangements and rearrangements with kappa deleting element (KDE). These rearrangements cannot generate functional TCR and BCR. Junctions of gene segments forming partial rearrangements similar to CDR3 have a high level of diversity due to random non-template insertions and deletions.

Adaptome (adaptive immunome, VDJ repertoire, immune repertoire) is defined as a set of all TCR and BCR loci rearrangements (complete and partial) identified in the sample. DNA adaptome or DNA-based adaptome can be referred to as an adaptome obtained using DNA as a template for sequencing library preparation.

### Methods For Detection of MRD

In aspects, disclosed herein is a method of determining the presence and quantification of minimal residual disease (MRD) in patients with hematological malignancies, the method comprising:
(a) detecting rearrangements of T cell receptor genes and B cell receptor genes characteristic for malignant clones in an initial sample from a patient using:
   a multiplex polymerase chain reaction (PCR) with isolated genomic DNA wherein the PCR reaction comprises a library of oligonucleotides amplifying one or more of the following: TRα/β/γ/δ, IgH/K/λ, DJ, DD, VD, and kappa-deleting element (KDE) rearrangements in a single multiplex mixture of oligonucleotides;
   high-throughput sequencing of the obtained PCR products; extracting complete and incomplete rearrangements by mapping potential rearrangements to a library comprising V, D, and J gene segments, kappa deletion element (KDE), and IGKC intron sequences, wherein the extraction is performed using a semi-global alignment algorithm to identify flanking sequences, followed by a clustering algorithm correcting for PCR and sequencing errors to assemble complete and/or incomplete rearrangement clonotypes;
   determining precise clonotype sizes from hematological malignancy-related repertoire; and
   malignancy-related clonotype detection; and
(b) performing follow-up monitoring of MRD in at least 4 independent PCR reactions with isolated and quantified genomic DNA obtained at a follow-up time point; wherein the concentration of a malignant clone in the follow-up time point is determined based on the proportion of independent PCR reactions where the corresponding rearrangement characteristic for the malignant clone is observed.

In aspects of the method, the hematological malignancy is a leukemia, a lymphoma or a multiple myeloma. In aspects, the leukemia is Acute lymphocytic leukemia, Chronic lymphocytic leukemia, Chronic myeloid leukemia, Acute myeloid leukemia, Hairy cell leukemia, Acute promyelocytic leukemia, T-cell prolymphocytic leukemia, Lymphoid leukemia, Large granular lymphocytic leukemia, or B-cell prolymphocytic leukemia. In aspects, the lymphoma is Non-Hodgkin lymphoma, Hodgkin's lymphoma, Diffuse large B-cell lymphoma, Follicular lymphoma, Burkitt lymphoma, Mantle cell lymphoma, Waldenström macroglobulinemia, Peripheral T-cell lymphoma, B-cell lymphoma, Nodular lymphocyte predominant Hodgkin lymphoma, B-cell chronic lymphocytic leukemia, Cutaneous B-cell lymphoma, Angioimmunoblastic T-cell lymphoma or Central nervous system lymphoma. In aspects, the multiple myeloma is Light Chain Myeloma, Non-secretory Myeloma, Solitary Plasmacytoma, Extramedullary Plasmacytoma, Monoclonal Gammopathy of Undetermined Significance, Smoldering Multiple Myeloma, Immunoglobulin D (IgD) Myeloma or Immunoglobulin E (IgE) Myeloma.

In aspects of the method, the method is performed at the onset of the hematological malignancy in the patient. In aspects, the method is performed during a relapse of the hematological malignancy in the patient. In aspects, the method is performed following treatment of the hematological malignancy. In aspects, the method is performed during treatment of the hematological malignancy. In aspects, the method is performed when the patient is thought to be in remission as determined by a cancer scan or standard cancer laboratory test.

In aspects of the method, the sample is bone marrow, blood, plasma, tissue (including formalin-fixed paraffin-embedded (FFPE) tissue samples). DNA may be isolated from cells or may be cell-free DNA.

In aspects of the method, extracting the complete and incomplete (i.e., partial) rearrangements is performed using a semi-global alignment (or glocal) algorithm. In general, a semi-global alignment is a variant of global alignment that allows for gaps at the beginning and/or the end of one of the sequences. Semi-global alignment algorithms search for the best possible alignment between sequences. Semi-global alignment algorithms known in the art can be used in the methods described herein. In aspects, the semi-global alignment algorithm is MiXCR as described in Bolotin et al. (Nat Methods 12, 380-381 (2015)//doi.org/10.1038/nmeth.3364).

In aspects of the method, precise clonotype sizes are determined using non-functional (e.g., non-productive or out-of-frame) clonotypes for quantitative bias correction. Non-functional clonotypes arise from non-functional rearrangements. As out-of-frame or stop codon containing TCR/BCR rearrangements do not form a functional receptor, they are not subjected to any specific clonal expansions and selection (Murugan et al., 2012, PNAS 109:16161-16166). As a passenger genomic variation, these rearrangements change their initial (recombinational) clonal frequencies randomly following the frequency changes of the second functional (in-frame) TCR/BCR allele present in the same T/B cell clone. According to the TCR/BCR loci rearrangement mechanism, the formation of in-frame and out-of-frame allele combinations in the same cell is also a stochastic and independent process in terms of V- and J-genes frequency. Thus, V- and J-gene frequencies among out-of-frame rearrangements must be sufficiently stable and must be equal to the initial recombination frequencies. Reproducible deviation of out-of-frame V- and J-gene frequencies (for the same multiplex PCR primer set) from the initial recombinational frequencies observed in the sequenced repertoire dataset is determined to be a result of artificial aberration caused by PCR amplification rather than immune repertoire evolution. Thus out-of-frame clonotypes can be considered a natural calibrator that can be used to measure amplification bias and quantitatively correct immune repertoire data. Examples of algorithms that can perform this quantitative bias correction are known in the art. In aspects, the quantitative bias correction is performed using the multiplex PCR-specific bias evaluation and correction algorithm iROAR: immune Repertoire Over Amplification Removal (github.com/smiranast/iROAR; Smirnova et al., 2023, eLife 12:e69157). In other aspects, the quantitative bias correction is performed using a DNA spike in method such as that described by Carlson et al. (Nat Commun 4, 2680 (2013), doi.org/10.1038/ncomms3680).

In aspects of the method, follow-up monitoring is performed at a follow-up timepoint. In aspects, the follow-up timepoint is about 1, 2, 3, 4, 5, 6, 7, or 8 weeks following the initial detection. In aspects, the follow-up timepoint is about 1, 2, 3, 4, 5, 6, 7, 8, 9, 10, 11 or 12 months following the initial detection. In aspects, the follow-up timepoint is about 1, 2 or 3 years following the initial detection.

In aspects of the method, follow-up monitoring is performed in at least 4 independent PCR reactions. In aspects, follow-up monitoring is performed in at least 6 independent PCR reactions. In aspects, follow-up monitoring is performed in at least 8 independent PCR reactions. In aspects, follow-up monitoring is performed in at least 10 independent PCR reactions. In aspects, follow-up monitoring is performed in at least 12 independent PCR reactions. In aspects, follow-up monitoring is performed in at least 14 independent PCR reactions. In aspects, follow-up monitoring is performed in at least 16 independent PCR reactions.

In aspects of the method, the minimal frequency of a malignant clone in the follow-up time point is determined based on the proportion of independent PCR reactions where the corresponding rearrangement characteristic for the malignant clone is observed. In aspects, the minimal frequency is determined as follows: the number of positive signals is divided by the number of analyzed diploid human genomes, whereas the number of analyzed diploid human genomes is calculated as total amount of analyzed DNA in all analyzed samples divided by 6.5 pg—the weight of one diploid human genome.

Without wishing to be bound by theory, in aspects of the method, the number of aliquots containing malignancy-related clonotypes is roughly comparable to the number of detected malignant cells. Thus, in aspects of the method, it is possible to count positive and negative aliquots. The determined portion of positive aliquots is then estimated to be the portion of the total cells assayed that are detected as malignant cells.

In aspects, the method further comprises determining the presence and minimal number of detected malignant cells in the patient if the corresponding rearrangement characteristic for the malignant clone is observed in all independent PCR reactions.

In aspects, the methods disclosed herein use a designed library of oligonucleotides for multiplex PCR. In aspects, the library of oligonucleotides for multiplex PCR is designed to decrease primer dimer formation and minimize nucleotide diversity.

In aspects, the oligonucleotide library is designed by a method comprising:
a. dividing target genomic DNA regions into k-mers of a certain length;
b. clustering the target genomic DNA regions using Hamming distances not exceeding 4, not exceeding 5, or not exceeding 6, or not exceeding 7 nucleotides; and
c. identifying clusters of highly similar k-mers according to the selected Hamming distance.

In aspects, the k-mers have a length of at least 16 bases. In aspects, the k-mers have a length of at least 12, 13, 14, 15, 16, 17, 18, 19, 20, 21, 22, 23, 24 or 25 bases.

In aspects, tables of 5-mers in the reverse orientation and 5-mers at the end of 3′ end of the nucleotide sequences in the forward orientation are made from the clusters identified. In aspects, the 5-mers are intersected pairwise, and the primer set with the lowest overlap with the 5-mers is selected for further analysis. In aspects, this intersection procedure is repeated. In aspects, the intersection procedure is performed in an iterative manner.

In aspects of the method, after a set of primers is determined, to equilibrate the annealing temperature of the primers, up to 14 additional target-site-related nucleotides are added to the 5′-end of the k-mers. In aspects, up to 12, 13, 14, 15, 16, 17, 18, 19 or 20 additional target-site-related nucleotides are added to the 5′-end of the k-mers.

In aspects of the method, 2 working sets of forward primers and 2 working sets of reverse primers are generated. In aspects, 3 working sets of forward primers and 3 working sets of reverse primers are generated. In aspects, 4 working sets of forward primers and 4 working sets of reverse primers are generated.

In aspects of the method, the reverse primer set comprises primers for J-genes, downstream introns for D-genes and KDE. In aspects, the forward primer set comprises primers for V-genes, upstream introns for D-genes and C-intron for IgK.

In aspects of the method, the multiplex primer sets generated can be used for amplification of complete VJ and/or VDJ rearrangements at one or more of TCRα, TCRβ, TCRγ, TCRδ, IgH, IgK, IgL loci; partial DJ rearrangements at TCRβ, TCRδ, IgH loci; VD and DD rearrangements at TCRδ and TCRβ loci; chimeric TRDV-TRAJ rearrangements; or Kappa deletion rearrangements. In aspects, the multiplex primer sets generated can be used for amplification of complete VJ and/or VDJ rearrangements at two, three, four, five, six, seven eight, nine, ten or more of TCRα, TCRβ, TCRγ, TCRδ, IgH, IgK, IgL loci; partial DJ rearrangements at TCRβ, TCRδ, IgH loci; VD and DD rearrangements at TCRδ and TCRβ loci; chimeric TRDV-TRAJ rearrangements; or Kappa deletion rearrangements. In aspects of the method, the multiplex primer sets generated can be used for amplification of complete VJ and/or VDJ rearrangements at each of TCRα, TCRβ, TCRγ, TCRδ, IgH, IgK, IgL loci; partial DJ rearrangements at TCRβ, TCRδ, IgH loci; VD and DD rearrangements at TCRδ and TCRβ loci; chimeric TRDV-TRAJ rearrangements; or Kappa deletion rearrangements.

In aspects of the method, primers for a particular V, D and J combination can be used to reduce unnecessary background for more specific amplification of target malignancy-related rearrangements and to reduce required sequencing depth (coverage).

In aspects of the method, indexing or dual indexing of each aliquot amplicon and introduction of adapters for sequencing are performed using “step-out” PCR. Step-out PCR is discussed in Wesley et al., (1997). Rapid Directional Walk Within DNA Clones by Step-Out PCR. In: White, B.A. (eds) PCR Cloning Protocols. Methods in Molecular Biology™, vol 67. Humana Press, Totowa, NJ.

In aspect of the method, indexing or dual indexing of each aliquot amplicon and introduction of adaptors for sequencing are performed using known in the art ligation reaction (Head at all., Library construction for next-generation sequencing: overviews and challenges. BioTechniques, 2014 Feb. 1;56(2):61-4. doi:10.2144/000114133).

In aspects of the methods, the amplicons are sequenced using next-generation sequencing methods. Such methods are known in the art. In aspects, single side sequencing is used. In aspects, double side sequencing is used. In aspects, single side sequencing used can be at least 100 nt, at least 150 nt, at least 250 nt, or at least 300 nt length. In aspects, double side sequencing used can be at least 50 nt, at least 100 nt, at least 150 nt, at least 250 nt length. In aspects, first side sequencing length and second side sequencing length may differ. In aspects, sequencing coverage of 1, 2, 3 and more sequencing reads per cell (per each 6.5 pg of genomic DNA) in the analyzed sample is used to detect TCR and BCR rearrangements presented in the prepared adaptome library.

In aspects of the methods, follow-on monitoring is performed with less than the full set of oligonucleotides from the initial multiplex detection. In aspects, the follow-on monitoring PCR reactions comprises one or more, two or more, three or more, four or more, five or more or six or more oligonucleotides amplifying TRα/β/γ/δ, IgH/K/λ, DJ, DD, VD, and kappa-deleting element (KDE) rearrangements in a single multiplex mixture of oligonucleotides.

In aspects of the methods, the average length of target amplicons is from about 120 bp to about 280 bp. In aspects, the average length of target amplicons is about 100, 110, 120, 130, 140, 150, 160, 170, 180, 190, 200, 210, 220, 230, 240, 250, 260, 270, 280, 290 or 300 bp. In aspects of the methods, the average lengths of target amplicons in the 4 multiplex primer sets are: set 113 about 120 bp, set 2—about 160 bp, set 3—about 240 bp, and set 4—about 280 bp.

In aspects of the methods, amplicons obtained with a set of primers cannot be a matrix for PCR with the subsequent set of primers. In aspects, the fact that a set of primers cannot be a matrix for PCR with the subsequent set of primers can prevent contaminating sequences in the sample from being further amplified. In aspects, the 1^{st} set of primers cannot be a matrix for PCR with the 2^{nd} set of primers. In aspects, the 1^{st} set of primers cannot be a matrix for PCR with the 2^{nd} set of primers or the 3rd set of primers. In aspects, the 1^{st} set of primers cannot be a matrix for PCR with the 2^{nd} set of primers, 3rd set of primers or 4th set of primers. In aspects, the 2^{nd} set of primers cannot be a matrix for PCR with the 3rd set of primers. In aspects, the 2^{nd} set of primers cannot be a matrix for PCR with the 3rd set of primers or 4th set of primers. In aspects, the 3^{rd} set of primers cannot be a matrix for PCR with the 4th set of primers.

In aspects of the methods where there are more than four sets of primers, the set of primers for a PCR reaction cannot be a matrix for the directly following PCR reaction.

In aspects of the methods, the concentrations of primers in the multiplex mixture are optimized. In aspects, the concentrations of primers in the multiplex mixture are optimized by analyzing the frequency ratios of non-functional rearrangements of T- and B-cell receptor genes.

In aspects the primer concentration is optimized by determining the overamplification rate (OAR) and adjusting the primer concentration to compensate for the OAR. In aspects, the OAR is determined using the method in Example 7 below.

In aspects of the methods, a ratio of weighted frequencies (proportion of reads) to unweighted frequencies (proportion of clonotypes) for V, D and J genes is determined. In aspects, if the ratio is a substantial deviation from 1, it is indicative of quantitative bias occurring during amplification. In aspects, the concentrations of primers in the multiplex mixture is selected to minimize the deviation from the expected value of one for the ratio of weighted frequencies to unweighted frequencies.

In aspects of the methods, the MRD detection sensitivity depends exclusively on the available input DNA and sequencing coverage.

In other aspects, the disclosure provides a method of determining the presence and quantification of Minimal residual disease (MRD) level in patients with hematological malignancies, the method comprising:
a. detecting rearrangements of T cell receptor genes and/or B cell receptor genes characteristic for a malignant clones in an initial sample from a patient using a multiplex polymerase chain reaction (PCR) with isolated genomic DNA and using high-throughput sequencing to obtained a PCR library; wherein the PCR reaction comprises oligonucleotides amplifying one or more of the following: TCRα/β/γ/δ, IgH/k/λ, DJ, DD, VD, and kappa deleting element (KDE) rearrangements, in a single multiplex mixture of oligonucleotides;
b. extracting complete and incomplete rearrangements from high-throughput sequencing data by mapping potential rearrangements to a library comprising V, D, and J gene segments, kappa deleting element (KDE), and IGKC intron RSS sequences, wherein the extracting is performed using a semi-global alignment algorithm to identify flanking sequences, followed by a clustering algorithm correcting for PCR and sequencing errors to assemble complete and/or incomplete rearrangement clonotypes;
c. determining precise clonotype sizes from hematological malignancy-related VDJ repertoire data using non-functional clonotypes as a calibrator for quantitative bias correction;
d. performing follow-up monitoring of MRD in at least 4 independent PCR reactions with isolated and quantified genomic DNA obtained at a follow-up time point; wherein the minimal frequency of a malignant clone in the follow-up time point is determined based on the proportion of independent PCR reactions where the corresponding rearrangement characteristic for the malignant clone is observed.

### Methods of Designing of Oligonucleotide Libraries

In aspects, the disclosure also provides a method for generating a library of oligonucleotides for multiplex PCR. In aspects, the library of oligonucleotides for multiplex PCR is designed to decrease primer dimer formation and minimize nucleotide diversity.

In aspects, the oligonucleotide library is designed by a method comprising:
a. dividing target genomic DNA regions into k-mers of a certain length;
b. clustering the target genomic DNA regions using Hamming distances distances not exceeding 4, not exceeding 5, or not exceeding 6, or not exceeding 7 nucleotides; and
c. identifying clusters of highly similar k-mers according to the selected Hamming distance.

In aspects, the k-mers have a length of at least 16 bases. In aspects, the k-mers have a length of at least 12, 13, 14, 15, 16, 17, 18, 19, 20, 21, 22, 23, 24 or 25 bases.

In aspects, tables of 5-mers in the reverse orientation and 5-mers at the end of 3′ end of the nucleotide sequences in the forward orientation are made from the clusters identified. In aspects, the 5-mers tin the tables are intersected pairwise, and the primer set with the lowest overlap with the 5-mers is selected for further analysis. In aspects, this intersection procedure is repeated. In aspects, the intersection procedure is performed in an iterative manner.

In aspects of the method, after a set of primers is determined, to equilibrate the annealing temperature of the primers, up to 14 additional target-site-related nucleotides are added to the 5′-end of the k-mers. In aspects, up to 12, 13, 14, 15, 16, 17, 18, 19 or 20 additional target-site-related nucleotides are added to the 5′-end of the k-mers.

In aspects of the method, 2 working sets of forward primers and 2 working sets of reverse primers are generated. In aspects, 3 working sets of forward primers and 3 working sets of reverse primers are generated. In aspects, 4 working sets of forward primers and 4 working sets of reverse primers are generated.

In aspects, the forward primer set comprises primers for V-genes, upstream introns for D-genes and C-intron for IgK. In aspects of the method, the reverse primer set comprises primers for J-genes, downstream introns for D-genes and KDE.

In aspects of the method, the multiplex primer sets generated can be used for separate amplification of complete VJ and/or VDJ rearrangements at TRα, TRβ, TRγ, TRδ, IgH, IgK, IgL loci; partial DJ rearrangements at TRβ, TRδ, IgH loci; VD and DD rearrangements at TRδ and TRβ loci; VD rearrangements at IgH loci; chimeric TRDV-TRAJ rearrangements; or Kappa deletion rearrangements.

In aspects of the method, primers for a particular V, D and J combination can be used to reduce unnecessary background for more specific amplification of target malignancy-related rearrangements and to reduce required sequencing coverage.

It is to be understood that the disclosure is not limited to the particular aspects exemplified below, as variations of the particular aspects may be made and still fall within the scope of the appended claims. It is also to be understood that the terminology employed is for the purpose of describing particular aspects, and is not intended to be limiting.

### Examples

### Example 1. Nested Localization of Oligos in Sequential MRD Detection and Follow up Time Points.

This example provides a scheme of annealing positions for four designed primer sets: forward primers set1, set2 and reverse primers set3, set4.

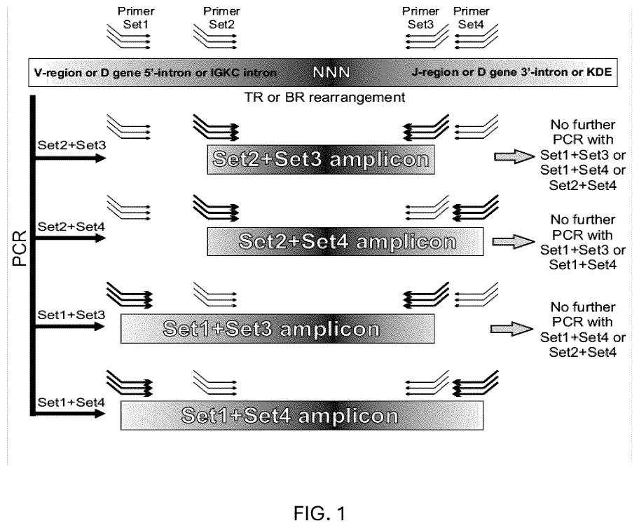

FIG. 1 is a schematic showing how nested localization of different primer sets is used to avoid undesired amplification of TCR/BCR amplicons obtained in the previous stage of MRD monitoring, for example during clonal marker detection in the onset sample. The amplicon is rich in molecules with target clonal markers so it might be one of the main sources of contamination, leading to potential false positive MRD results. The proposed primer positions are reliable protection against such cross-contamination.

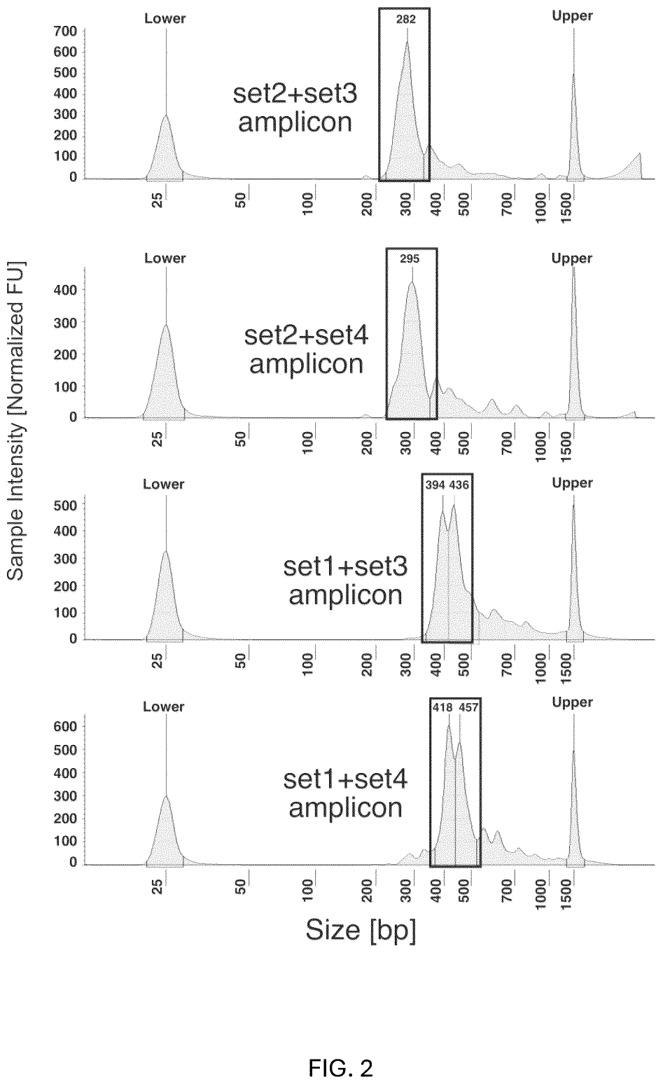

FIG. 2 shows picks of four different tested target amplicons as separated using capillary electrophoresis (Tape Station, Agilent). All four picks correspond to ready-to-sequence TCR/BCR libraries differ one from another by amplicon length. As can be seen from the figure, all four combinations of forward and reverse primer sets can be used for adaptome library generation.

### Example 2. Validation of Multiplex Primer Mix

Primer mixes capable of amplifying multiple types of VDJ rearrangements were prepared. One example of such a primer mix is the 7GENES mixture sold by MiLaboratories, Inc. To validate the efficiency of the multiplex mix to amplify diverse VDJ rearrangements, a single tube 7GENES (set1+set3 primers) amplification was performed starting from 100-150 ng of human PBMC DNA derived from 190 healthy donors. Amplification was performed separately for each donor. Detections were performed for IGHV, IGHD, IGKV, IGLV, TRBV and TRBD, TRGV, TRDV+TRDD, IGHJ, IGKJ, IGLJ, TRAJ, TRBJ, and TRDJ gene segments.

The resulting libraries were sequenced on an Illumina NextSeq550 sequencer using paired-end 150+150 nt sequencing, and analyzed using MiXCR software (/pubmed.ncbi.nlm.nih.gov/25924071/) with additional reference datasets required to extract incomplete rearrangements.

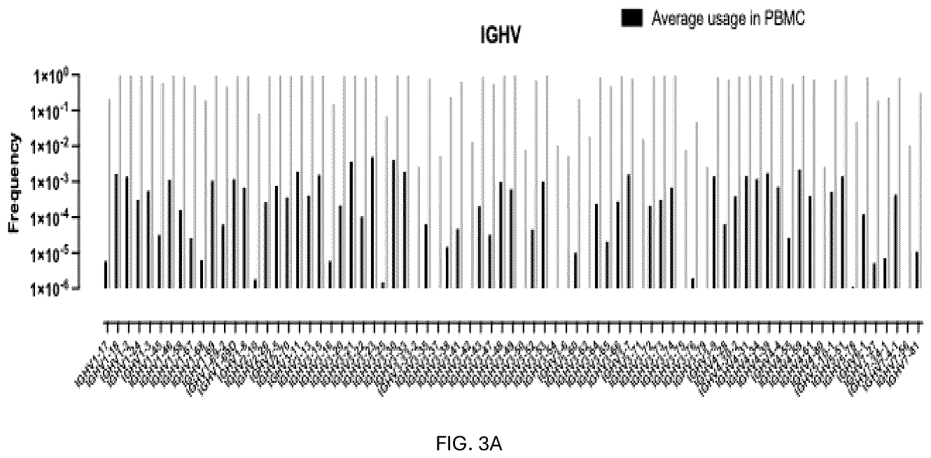

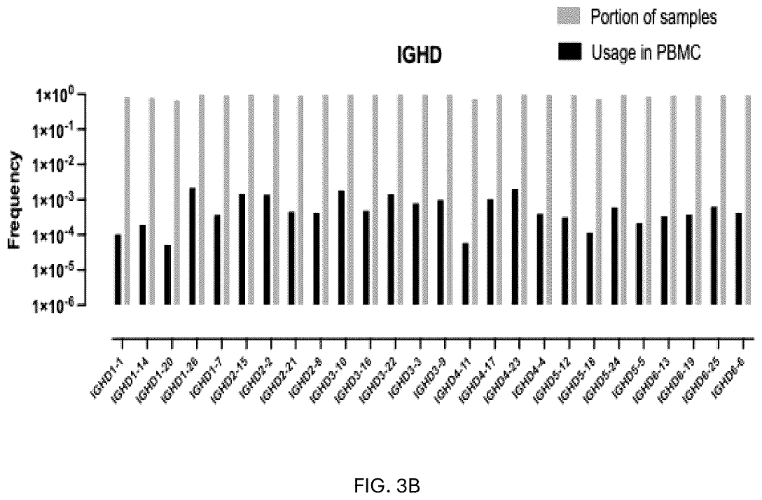

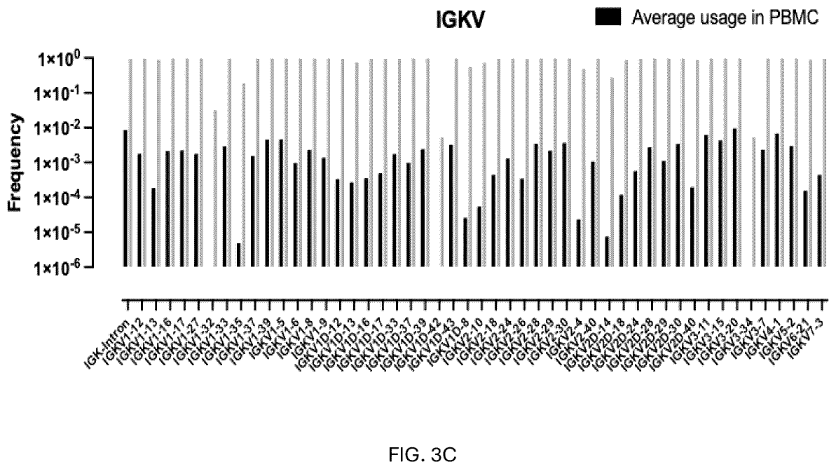

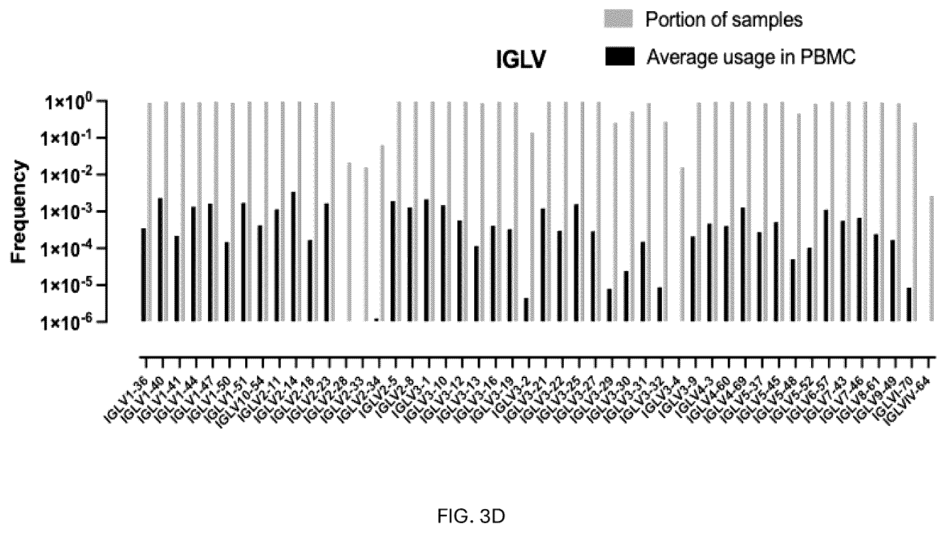

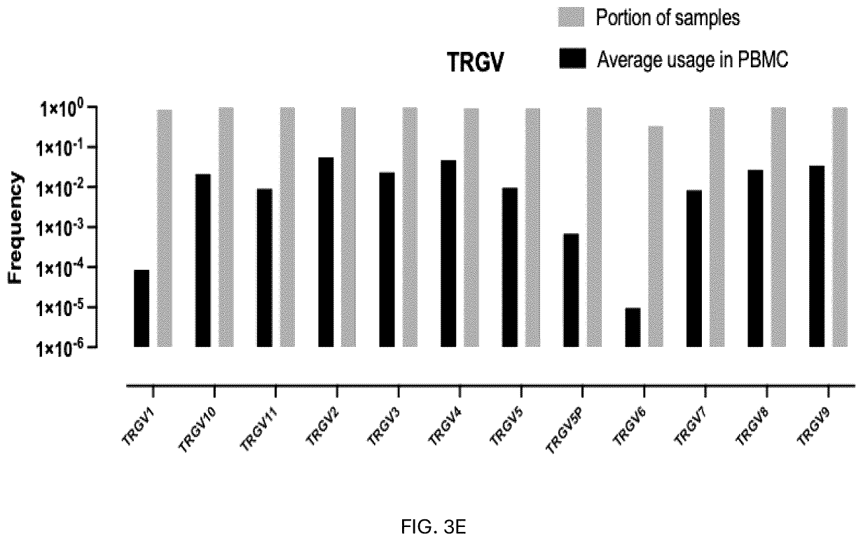

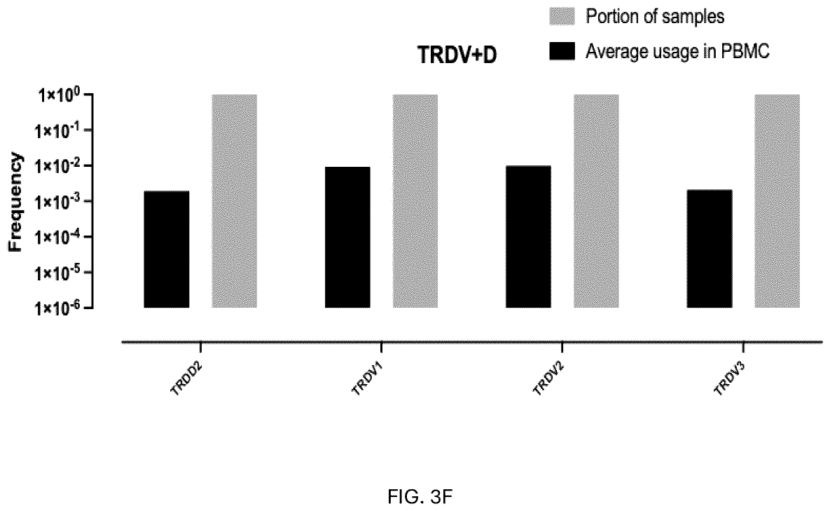

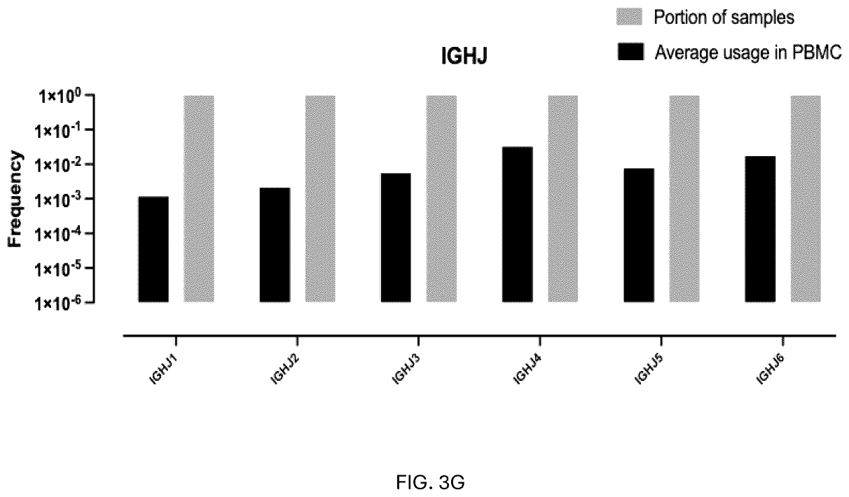

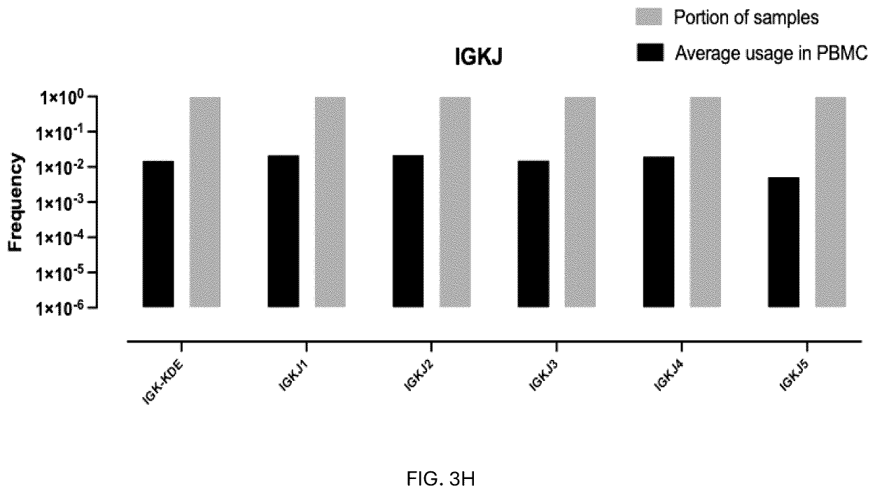

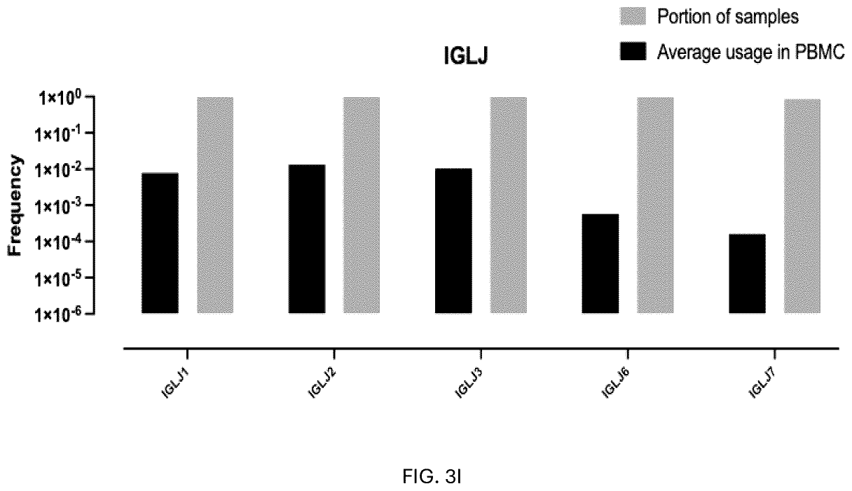

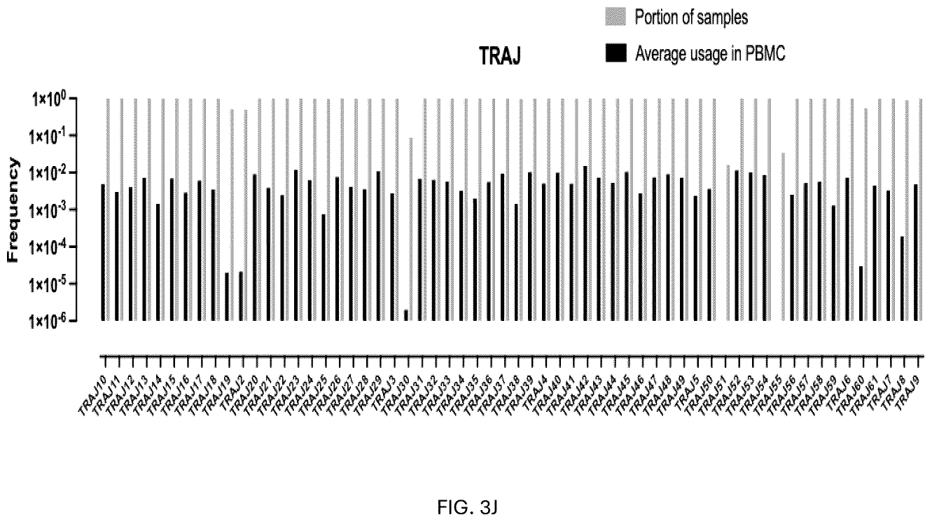

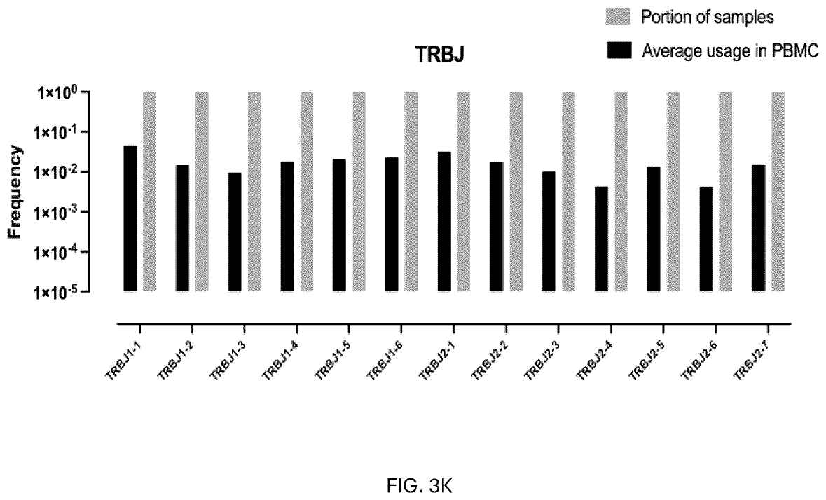

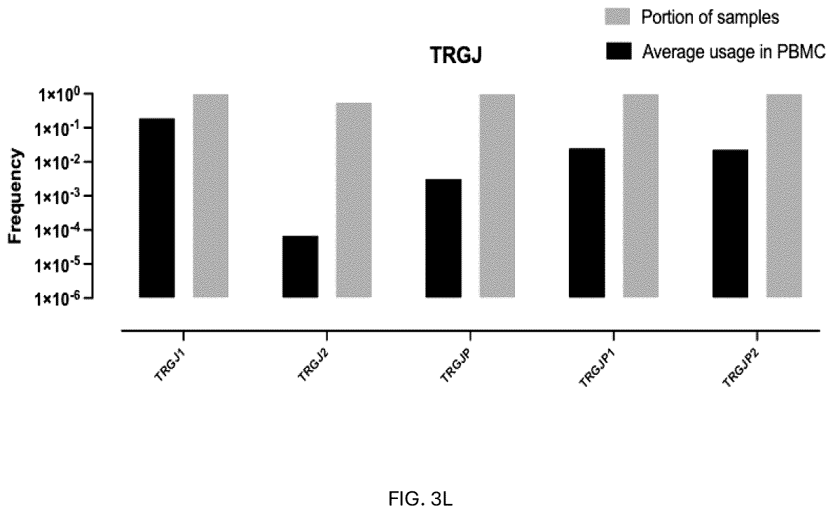

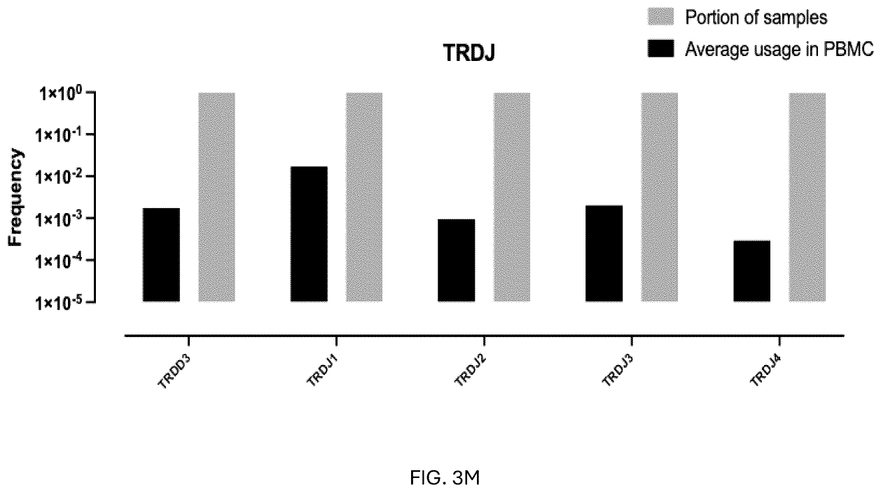

FIGS. 3A-M show the gene segment frequency in complete and incomplete rearrangements and the proportion of samples containing rearrangements with such gene segments across 190 healthy donors.

A total of 9,532,826 unique rearrangements with 5619 combinations of 296 V-genes, 29 D-genes, Kde, IGKC-intron and 97 J-genes were identified in DNA from the peripheral blood of 190 healthy individuals, confirming the successful performance of the designed multiplex primers.

The results demonstrate that the designed multiplex primers are capable of amplifying the desired gene rearrangements and provide nearly comprehensive coverage of adaptome rearrangements for their initial detection and MRD follow up.

### Example 3. Validation of Method Using a Diluted Spike-in Leukemic Clone Carrying Several VDJ Rearrangements

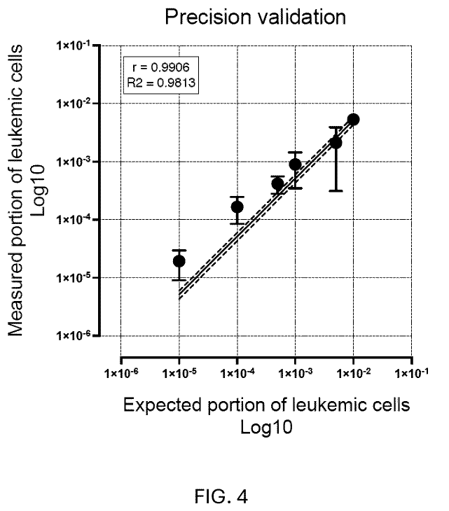

This example demonstrates the precision of MRD detection based on the approach presented in this disclosure. To measure precision, observed and expected MRD levels (concentration of leukemic cells) were compared in six composite samples containing a counted number of cells of regenerating bone marrow (background cells) from one patient and a counted number of leukemic cells from another patient (spike-in cells). Cell counting and mixing were performed using FACS (Fluorescence Activated Cell Sorting). Spike-in leukemic cells (10, 100, 500, 1000, 5000 and 10 000 cells) were sorted into separate aliquots (1 mln cells each) of background cells to imitate MRD at six different levels: 1 leukemic cell per 1) 100 000 cells (0.001% MRD); 2) 10 000 cells (0.01% MRD); 3) 5000 cells (0.02% MRD), 4) 1000 cells (0.1% MRD) 5) 500 cells (0.2% MRD); 6) 100 cells (1% MRD). DNA from the obtained mixtures was extracted and used for sequencing library preparation. The obtained DNA for each spike-in sample was distributed among 32 separate multiplex PCR reactions with a 320 ng DNA per reaction for 0.001% MRD, a 32 ng DNA per reaction for 0.01% MRD, 16 ng DNA per reaction for 0.02% MRD, a 3.2 ng DNA per reaction for 0.1% MRD, a 1.6 ng DNA per reaction for 0.2% MRD, and a 0.32 ng DNA per reaction for 1% MRD. Set1+Set4 primers were used for PCR to avoid target amplicon contamination by any previous PCR products. Three different rearrangements (IGH D-J, TRD D2-D3, TRD V2-D3) initially detected in pure leukemia sample were used as clonal markers to track leukemic cell presence in prepared test samples. A plot of the measured portion of leukemic cells vs. the expected portion of leukemic cells is shown in FIG. 4. As can be seen in the figure, the method showed high precision, with r=0.9906 and R2=0.9813. This experiment shows that the method can be used for high precision determination of malignant cells in a sample using several VDJ rearrangements.

Example 4. Comparison of NGS Analysis with Flow-Cytometry Using Real Clinical Data

This example demonstrates the level of consistency between the disclosed Next-generation sequencing (NGS) based approach and the conventional flow cytometry-based MRD monitoring method.

Multicolor flow cytometry for MRD measurement was performed using 8-12 surface antigens according to the consensus recommendations of the AIEOP-BFM group [M. Dworzak et. al., Standardization of flow cytometric minimal residual disease evaluation in acute lymphoblastic leukemia: Multicentric assessment is feasible. Cytometry B Clin Cytom. 2008 November;74(6):331-40. doi: 10.1002/cyto.b.20430]. Identification of CD19-negative relapses during CD19-directed immunotherapy was performed using multicolor flow cytometry with a modified antibody panel and adjusted data analysis algorithm, according to the recommendations of Cherian et al. [Cherian et al. A novel flow cytometric assay for detection of residual disease in patients with B-lymphoblastic leukemia/lymphoma post anti-CD19 therapy. Cytometry B Clin Cytom. 2018 January;94(1):112-120. doi: 10.1002/cyto.b.21482. Epub 2016 Sep. 23.].

Libraries for NGS-based MRD analysis were prepared using forward and reverse primers from set1 and set4, specific for V/D/J/Kde combinations present in pre-detected leukemic clone-related rearrangements. A total of 32 DNA aliquots (16 for 320 ng, 8 for 32 ng, 8 for 3.2 ng DNA) were distributed across separate multiplex PCR reactions for each analyzed time point. Amplicons from each of the 32 PCR reactions were indexed uniquely in the subsequent indexing PCR. Pooled libraries for each time point were sequenced on the MiSeq instrument in paired-ends mode 150+150 nt and ˜1 mln sequencing reads per time-point.

Results are provided in Table 1 below. As can be seen from the results, the specificity and sensitivity of the NGS-based method are similar to flow-cytometry methods and, furthermore, in manycases, the NGS-based method is able to detect MRD that flow-cytometry is not.

TABLE 1
Number of positive and negative MRD results obtained by disclosed
NGS-based approach and conventional flow cytometry.
58 patients (386 time-points)

NGS

| | | MRD positive | MRD negative |
|---|---|---|---|
| Flow | MRD positive | 74 | 12 |
| cytometry | MRD negative | 64 | 236 |

### Example 5. Classic and Non-Classic Examples of VDJ Rearrangements in Leukemic Cells

This example provides examples of classic and non-classic TCR and BCR rearrangements which are used as patient-specific clonal biomarkers for MRD monitoring.

The presented rearrangements were detected in DNA from actual clinical diagnostic samples of bone marrow from T-cell acute lymphoblastic leukemia and B-cell acute lymphoblastic leukemia patients. The detection was performed using the 7GENES kit (a mix of set1 and set3 primers).

Classic VDJ rearrangements that are detected include: partial rearrangements between D and J genes (DJ rearrangements) in TRB, TRD and IGH loci; partial rearrangements between V and D, D and D genes in TRD locus; rearrangements between IGK C-intron or IGKV genes and Kappa deletion element (Kde); complete rearrangements between V, D and J genes in TRB, TRD and IGH loci and rearrangements between V and J genes in TRA, TRG, IGK and IGL loci.

Non-classic VDJ rearrangements that are detected include: partial rearrangements between D1 and D2 genes in TRB locus; partial rearrangements between V and D genes in TRB and IGH loci; complete chimeric rearrangements between TRDV genes and TRAJ genes. The designed multiplex system contains primers for all TCR and BCR genes in the single common mix which allow detection of all chimeric VDJ combinations. Examples of specific rearrangements that can be detected are shown in Tables 2-18 below.

TABLE 2
Examples of TRA V-J rearrangements

| | Sequence of | SEQ | | | V | J |
|---|---|---|---|---|---|---|
| Clone | hypervariable | ID | TRAV | TRAJ | gene | gene |
| size | region | NO. | gene | gene | end | start |
| 0.72 | TGTGCAGCACTCTAA | 1 | V29 | J48 | 8 | 10 |
| | CTTTGGAAATGAGAA | | | | | |
| | ATTAACCTTT | | | | | |
| 0.50 | TGTGCCAGTGAACTC | 2 | V17 | J41 | 4 | 9 |
| | AAATTCCGGGTATGC | | | | | |
| | ACTCAACTTC | | | | | |
| 0.67 | TGTGCCCAGAACTAA | 3 | V38-2 | J48 | 4 | 11 |
| | CTTTGGAAATGAGAA | | | | | |
| | ATTAACCTTT | | | | | |
| 0.51 | TGTGCTTCAGAGGCG | 4 | V6 | J4 | 5 | 14 |
| | TTTTCTGGTGGCTAC | | | | | |
| | AATAAGCTGATTTTT | | | | | |

TABLE 3
Examples of TRB D-J rearrangements

| | Sequence of | SEQ | | | D | J |
|---|---|---|---|---|---|---|
| Clone | hypervariable | ID | TRBD | TRBJ | gene | gene |
| size | region | NO. | gene | gene | end | start |
| 0.94 | GGGACAGGGGGGGGA | 5 | D1 | J2-5 | 10 | 14 |
| | GAGACCCAGTACTTC | | | | | |
| | GGGCC | | | | | |
| 0.99 | GGGACTCCTACGAGC | 6 | D2 | J2-7 | 6 | 7 |
| | AGTACGTCGGGCC | | | | | |
| 0.88 | GGGACTGTCCGCGCA | 7 | D2 | J2-7 | 5 | 14 |
| | CGAGCAGTACTTCGG | | | | | |
| | GCC | | | | | |
| 0.56 | GGGACAGCAAATGTC | 8 | D1 | J2-3 | 6 | 30 |
| | ACCCGAAGTTAAGAT | | | | | |
| | CACAGATACGCAGTA | | | | | |
| | TTTTGGCCC | | | | | |

TABLE 4
Examples of TRB VDJ rearrangements

| | Sequence of | SEQ | | | | | | | |
|---|---|---|---|---|---|---|---|---|---|
| Clone | hypervariable | ID | TRBV | TRBD | TRBJ | V | D | D | J |
| size | region | NO. | gene | gene | gene | end | start | end | start |
| 0.83 | TGTGCCAGCAGCT | 9 | V7-9 | D1 | J2-2 | 12 | 13 | 18 | 22 |
| | AGGGGGAGGGAAC | | | | | | | | |
| | ACCGGGGAGCTGT | | | | | | | | |
| | TTTTT | | | | | | | | |
| 0.62 | TGTGCCAGCAGCT | 10 | V5-5 | D2 | J2-2 | 12 | 17 | 22 | 25 |
| | GTCGAGCGGGGTC | | | | | | | | |
| | ACCGGGGAGCTGT | | | | | | | | |
| | TTTTT | | | | | | | | |
| 0.96 | TGTGCCAGCAGCT | 11 | V5-8 | D1 | J1-5 | 12 | 16 | 21 | 28 |
| | CCAAGGGGGATGA | | | | | | | | |
| | CGATCAGCCCCAG | | | | | | | | |
| | CATTTT | | | | | | | | |
| 0.87 | TGTGCCAGCAGCT | 12 | V6-5 | D2 | J2-1 | 10 | 13 | 20 | 21 |
| | CGGGACTAGAGCA | | | | | | | | |
| | CGTTCTTC | | | | | | | | |

TABLE 5
Examples of TRG V-J rearrangements

| | Sequence of | SEQ | | | V | J |
|---|---|---|---|---|---|---|
| Clone | hypervariable | ID | TRGV | TRGJ | gene | gene |
| size | region | NO. | gene | gene | end | start |
| 0.41 | TGTGCCTTGTGGGAGG | 13 | V9 | J1 | 17 | 22 |
| | TGGGAGGAAACTCTTT | | | | | |
| 0.74 | TGTGCCTGCTGGCCCC | 14 | V11 | J1 | 11 | 28 |
| | TCACTCTTCAGCATAA | | | | | |
| | GAAACTCTTT | | | | | |
| 0.53 | TGTGCCACCTGGGACG | 15 | V2 | J1 | 18 | 23 |
| | GGCGGAATATAAGAAA | | | | | |
| | CTCTTT | | | | | |
| 0.59 | TGTGCTGCGTGGGAT | 16 | V10 | J1 | 15 | 25 |
| | TCTCTTAATCGAAAC | | | | | |
| | TCTTT | | | | | |

TABLE 6
Examples of TRD V-D rearrangements

| | Sequence of | SEQ | | | V | D |
|---|---|---|---|---|---|---|
| Clone | hypervariable | ID | TRDV | TRDD | gene | gene |
| size | region | NO. | gene | gene | end | start |
| 0.61 | GTCTTACTACTGTGCC | 17 | V2 | D3 | 24 | 27 |
| | TGTGACACCTCACTGG | | | | | |
| | GGGATACGCA | | | | | |
| 1.00 | GTCTTACTACTGTGCC | 18 | V2 | D3 | 21 | 27 |
| | TGTGACCCCCAGGGAT | | | | | |
| | ACGCA | | | | | |
| 0.65 | GTCTTACTACTGTGCC | 19 | V2 | D3 | 21 | 30 |
| | TGTGACCACCCTTTGT | | | | | |
| | ACTGGGGGATACGCA | | | | | |
| 1.00 | GTCTTACTACTGTGCC | 20 | V2 | D3 | 26 | 32 |
| | TGTGACACCGGGAGAA | | | | | |
| | GGGGATACGCA | | | | | |

TABLE 7
Examples of TRD D-D rearrangements

| | Sequence of | SEQ | | | D2 | D3 |
|---|---|---|---|---|---|---|
| Clone | hypervariable | ID | TRDD | TRDD | gene | gene |
| size | region | NO. | gene | gene | end | start |
| 1.00 | ATGTGTTTCATTG | 21 | D2 | D3 | 20 | 28 |
| | TGCCTTCCCCCTC | | | | | |
| | CGGGGGGATACGC | | | | | |
| | A | | | | | |
| 1.00 | ATGTGTTTCATTG | 22 | D2 | D3 | 20 | 25 |
| | TGCCTTCCCGGGG | | | | | |
| | GGGGATACGCA | | | | | |
| 0.99 | ATGTGTTTCATTG | 23 | D2 | D3 | 20 | 24 |
| | TGCCTTCCCGGCC | | | | | |
| | CGTACTGGGGGAT | | | | | |
| | ACGCA | | | | | |
| 0.97 | ATGTGTTTCATTG | 24 | D2 | D3 | 16 | 23 |
| | TGCCCCCTTCGGG | | | | | |
| | GGATACGCA | | | | | |

TABLE 8
Examples of TRD D-J rearrangements

| | Sequence of | SEQ | | | V | J |
|---|---|---|---|---|---|---|
| Clone | hypervariable | ID | TRDD | TRDJ | gene | gene |
| size | region | NO. | gene | gene | end | start |
| 0.98 | CATCCTGACTGGGG | 25 | D2 | J1 | 2 | 25 |
| | GCCCCCTTCCATAC | | | | | |
| | ACCGATAAACTCAT | | | | | |
| | CTTT | | | | | |
| 0.95 | CATTGTGCCTTCCC | 26 | D2 | J1 | 12 | 35 |
| | CCCGGGTCTGGGGG | | | | | |
| | ATCCGCCCACCGAT | | | | | |
| | AAACTCATCTTT | | | | | |
| 0.52 | CATTGTGCCTTCCC | 27 | D2 | J1 | 12 | 30 |
| | AACTGGGGGATACT | | | | | |
| | GGACCGATAAACTC | | | | | |
| | ATCTTT | | | | | |
| 0.99 | CATTGTGCCTTGCG | 28 | D2 | J1 | 10 | 24 |
| | GGGGGGATACATAA | | | | | |
| | ACTCATCTTT | | | | | |

TABLE 9
Examples of TRDV-TRAJ rearrangements

| | Sequence of | SEQ | | | TRDV | TRAJ |
|---|---|---|---|---|---|---|
| Clone | hypervariable | ID | TRDV | TRAJ | gene | gene |
| size | region | NO. | gene | gene | end | start |
| 0.87 | TGTGCCTGTGGCGG | 29 | V2 | J9 | 9 | 30 |
| | ATTACTGGGGGATC | | | | | |
| | CCAAATACTGGAGG | | | | | |
| | CTTCAAAACTATCT | | | | | |
| | TT | | | | | |
| 0.80 | TGTGCCTGTCAGTA | 30 | V2 | J61 | 8 | 24 |
| | CCCGGGGTCCACCG | | | | | |
| | GGTTAATAGGAAAC | | | | | |
| | TGACATTT | | | | | |
| 0.89 | TGTGCCGAACGTGC | 31 | V2 | J48 | 5 | 14 |
| | TCTAACTTTGGAAA | | | | | |
| | TGAGAAATTAACCT | | | | | |
| | TT | | | | | |
| 0.92 | TGTGCCTGTGACAC | 32 | | J29 | 14 | 33 |
| | CCCGAGGGGGGATA | | | | | |
| | AATAACAGGAAACA | | | | | |
| | CACCTCTTGTCTTT | V2 | | | | |

TABLE 10
Examples of IGH D-J rearrangements

| | Sequence of | SEQ | | | D | J |
|---|---|---|---|---|---|---|
| Clone | hypervariable | ID | IGHD | IGHJ | gene | gene |
| size | region | NO. | gene | gene | end | start |
| 0.81 | AGGATATTGTAGTAG | 33 | D2-2 | J1 | 24 | 70 |
| | TACCAGCTGCCGTCG | | | | | |
| | GCTACGGTGACTACG | | | | | |
| | TCCCCGGCTGAATAC | | | | | |
| | TTCCAGCACTGGGGC | | | | | |
| | CA | | | | | |
| 0.96 | ATATAACGATTTTTG | 34 | D3-3 | J3 | 18 | 31 |
| | GAGTTGAGGATGCTT | | | | | |
| | TTGATATATGGGGCC | | | | | |
| | A | | | | | |
| 0.9 | TGACTACGTTGGCT | 35 | D4-23 | J3 | 11 | 42 |
| | TTTGATATCTGGGG | | | | | |
| | CCA | | | | | |
| 0.96 | GTATTACTATGATAG | 36 | D3-22 | J6 | 25 | 30 |
| | TAGTGGTTATTTAAA | | | | | |
| | ATATTACTACTACTA | | | | | |
| | CTACGGTATGGACGT | | | | | |
| | CTGGGGCCA | | | | | |

TABLE 11
Examples of IGH V-D-J rearrangements

| | Sequence of | SEQ | | | | | | | |
|---|---|---|---|---|---|---|---|---|---|
| Clone | hypervariable | ID | IGHV | IGHD | IGHJ | V | D | D | J |
| size | region | NO. | gene | gene | gene | end | start | end | start |
| 0.92 | TGTGCGAGAAGCTTA | 37 | V3- | D3- | J4 | 8 | 21 | 51 | 54 |
| | AGGGCCTATTACTAT | | 33 | 22 | | | | | |
| | GATAGTAGTGGTTAT | | | | | | | | |
| | TACTACGACACTTTG | | | | | | | | |
| | ACTACTGG | | | | | | | | |
| 0.95 | TGTGCGAGAGTACCC | 38 | V4- | D2- | J4 | 9 | 14 | 21 | 25 |
| | AGCTACCTGATGACT | | 34 | 15 | | | | | |
| | ACTGG | | | | | | | | |
| 0.84 | TGTGCGAGCTCTCCG | 39 | V1- | D6- | J4 | 7 | 14 | 30 | 40 |
| | TATAGCAGCAGCTGG | | 69D | 13 | | | | | |
| | TTTTCTTGGTACTAC | | | | | | | | |
| | TGG | | | | | | | | |
| 0.96 | TGTGCTATGGGCCTT | 40 | V3- | D6- | J5 | 4 | 17 | 27 | 28 |
| | TCTATAGCAGCTCGT | | 30 | 6 | | | | | |
| | ACAACTGGTTCGACC | | | | | | | | |
| | CCTGG | | | | | | | | |

TABLE 12
Examples of IGK V-J rearrangements

| | Sequence of | SEQ | | | V | J |
|---|---|---|---|---|---|---|
| Clone | hypervariable | ID | IGKV | IGKJ | gene | gene |
| size | region | NO. | gene | gene | end | start |
| 0.99 | TGTCAACAGTATGAT | 41 | V1D-33 | J4 | 14 | 19 |
| | CGATGCGCTCACTTT | | | | | |
| | C | | | | | |
| 0.36 | TGTCAACAGCTTAAT | 42 | V1-9 | J5 | 22 | 22 |
| | AGTTACCCGATCACC | | | | | |
| | TTC | | | | | |
| 0.90 | GGTCAACGGACTTAC | 43 | V1D-37 | J2 | 27 | 34 |
| | AATGCCCCTCCGGGG | | | | | |
| | GTTCT | | | | | |
| 0.96 | TGTCAACAGTATGAT | 44 | V1D-33 | J4 | 21 | 25 |
| | AATCTCCGGCTTC | | | | | |

TABLE 13
Examples of IGK C-intron-
Kde rearrangements

| | Sequence of | SEQ | | | V | J |
|---|---|---|---|---|---|---|
| Clone | hypervariable | ID | IGK | IGK | gene | gene |
| size | region | NO. | region | region | end | start |
| 0.56 | CCAGCTTTCCTGATA | 45 | C- | KDE | 14 | 17 |
| | GGCCCTAGTGGCAGC | | Intron | | | |
| | CCAGGGCGACTC | | | | | |
| 0.42 | CCAGCTTTCCCGGAG | 46 | C- | KDE | 10 | 15 |
| | CCCTAGTGGCAGCCC | | Intron | | | |
| | AGGGCGACTC | | | | | |
| 0.42 | CCAGCTTTCCTGATA | 47 | C- | KDE | 14 | 16 |
| | CCCTAGTGGCAGCCC | | Intron | | | |
| | AGGGCGACTC | | | | | |
| 0.91 | CCAGCTTTCCTGAGC | 48 | C- | KDE | 13 | 14 |
| | CCTAGTGGCAGCCCA | | Intron | | | |
| | GGGCGACTC | | | | | |

TABLE 14
Examples of IGK V-Kde rearrangements

| | Sequence of | SEQ | | | V | J |
|---|---|---|---|---|---|---|
| Clone | hypervariable | ID | IGKV | IGK | gene | gene |
| size | region | NO. | gene | region | end | start |
| 0.85 | AGTCCCGGGGAGTGG | 49 | V1D-43 | KDE | 5 | 11 |
| | CAGCCCAGGGCGACT | | | | | |
| | C | | | | | |
| 0.98 | ATGCCCGCCGGGTGG | 50 | V1-37 | KDE | 5 | 11 |
| | CAGCCCAGGGCGACT | | | | | |
| | C | | | | | |
| 0.52 | ACTGGCCCCGGCCCT | 51 | V2-30 | KDE | 6 | 16 |
| | AGTGGCAGCCCAGGG | | | | | |
| | CGACTC | | | | | |
| 0.69 | ATCTCCCGACGACGG | 52 | V1-33 | KDE | 6 | 14 |
| | GGCGACTC | | | | | |

TABLE 15
Examples of IGL V-J rearrangements

| | Sequence of | SEQ | | | V | J |
|---|---|---|---|---|---|---|
| Clone | hypervariable | ID | IGLV | IGLJ | gene | gene |
| size | region | NO. | gene | gene | end | start |
| 0.99 | TGCAGCTCATATGCA | 53 | V2-8 | J3 | 23 | 20 |
| | GGCAGCAACTGGGTG | | | | | |
| | TTC | | | | | |
| 1.00 | TGTCAATCAGCAGAC | 54 | V3-25 | J2 | 29 | 28 |
| | AGCAGTGGTACTTAT | | | | | |
| | GTGGTATTC | | | | | |
| 0.95 | TGCAGCTCATATGCA | 55 | V2-8 | J2 | 20 | 25 |
| | GGCAGCCTTTATGTG | | | | | |
| | GTATTC | | | | | |
| 0.96 | TGTCAGGCGTGGGAC | 56 | V3-1 | J1 | 28 | 26 |
| | AGCAGCACTGCATGT | | | | | |
| | CTTC | | | | | |

TABLE 16
Examples of human TRB V-D rearrangements
Human TRB V-D rearrangements

| | Sequence of | SEQ | | | V | D |
|---|---|---|---|---|---|---|
| Clone | hypervariable | ID | TRBV | TRBD | gene | gene |
| size | region | NO. | gene | gene | end | start |
| 0.99 | CTTCTACATCTGCAG | 57 | V20-1 | D2 | 22 | 23 |
| | TGCTAGAGGGACTAG | | | | | |
| | CGGGAGGG | | | | | |
| 0.73 | TGTGTATTTCTGTGC | 58 | V3-2 | D2 | 27 | 31 |
| | CAGCAGCCAAGATTG | | | | | |
| | GGCGGGGGGG | | | | | |
| 0.96 | CTTCTACATCTGCAG | 59 | V20-1 | D2 | 18 | 23 |
| | TGCTCTTAAGCGGGG | | | | | |
| | GGG | | | | | |
| 0.98 | TGTGTATTTCTGTGC | 60 | V3-2 | D2 | 28 | 33 |
| | CAGCAGCCAAGATCC | | | | | |
| | CGCCTGGCGGGGGGG | | | | | |

TABLE 17
Examples of human TRB D-D rearrangements.
Human TRB D-D rearrangements

| | Sequence of | SEQ | | | D1 | D2 |
|---|---|---|---|---|---|---|
| Clone | hypervariable | ID | TRBD | TRBD | gene | gene |
| size | region | NO. | gene | gene | end | start |
| 1.00 | GGGACAGGGGACTAG | 61 | D1 | D2 | 9 | 10 |
| | CGGGAGGG | | | | | |
| 0.91 | GGGACAGGGGGGTAG | 62 | D1 | D2 | 11 | 16 |
| | CTAGCGGGAGGG | | | | | |
| 0.88 | GGGACAGGGGGCGCC | 63 | D1 | D2 | 12 | 23 |
| | CCTGTTAGAGGG | | | | | |
| 0.99 | GGGACAGTTAAGGCT | 64 | D1 | D2 | 7 | 20 |
| | AGGGCTAGCGGGGGG | | | | | |
| | G | | | | | |

TABLE 18
Examples of human IGH V-D rearrangements.
Human IGH V-D rearrangements

| | Sequence of | SEQ | | | V | D |
|---|---|---|---|---|---|---|
| Clone | hypervariable | ID | IGHV | IGHD | gene | gene |
| size | region | NO. | gene | gene | end | start |
| 0.40 | TGTGCGAGAGCATAC | 65 | V3-7 | D6- | 9 | 25 |
| | CCTGAATCTTGGGTA | | | 19 | | |
| | TAGCAGTGGCTGGTA | | | | | |
| | C | | | | | |
| 1.00 | TGTGCACACCCCAGT | 66 | V2-5 | D6- | 8 | 11 |
| | GGCTGGTAC | | | 19 | | |
| 0.99 | TGTGCACACCGATGA | 67 | V2-5 | D4- | 8 | 11 |
| | CTACAGTAACTAC | | | 4 | | |
| 0.91 | TGTGCGAGTGTGAGT | 68 | V3-33 | D4- | 7 | 16 |
| | AACGGTGACTAC | | | 17 | | |

The results indicate that the NGS-based method is able to determine multiple, leukemic clone-specific, classic and non-classic examples of VDJ rearrangements that can be analyzed in follow-up samples for MRD analysis.

### Example 6. Analysis of Real Clinical Data on a Cohort of 58 B-ALL Patients

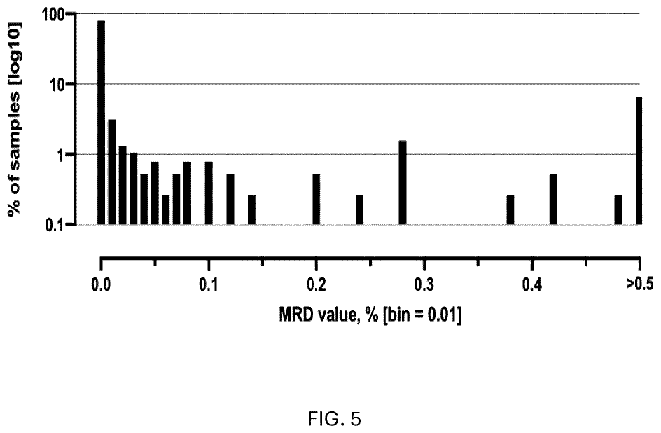

This example demonstrates detection and measurement (quantification) of MRD for actual clinical bone marrow samples obtained from acute leukemia patients during therapy. Observed patients were receiving anti-CD19 CAR-T cell therapy with subsequent bone marrow transplantation. MRD analysis for each patient was performed for 1-14 time points (6.5 average) for a total of 386 time-points. Timepoints included samples taken at 24 days, 2 months, 3 months, 6 months, 1 year, and 2 years after the onset of therapy. Quantification of MRD levels (concentration of leukemic cells) was performed by the technique described in this disclosure. A plot of the percentage of samples having the indicated MRD value is shown in FIG. 5.

The presence of target clonal rearrangements was analyzed by high throughput sequencing of VDJ libraries obtained from thirty-two independent DNA aliquots: 16 aliquots of 300 ng DNA, 8 aliquots of 30 ng DNA, and 8 aliquots of 3 ng DNA. Primary results of MRD marker extraction from VDJ repertoire data for one of the patients of the study are summarized in Table 19. The table contains the number of total sequencing reads obtained per aliquot and the number of reads covering target MRD markers in each analyzed aliquot.

Subsequent analysis of obtained primary results includes under-sequenced aliquot exclusion, removal contamination between aliquots, and calculation of MRD level. Under-sequencing is considered a source of false-negative results. Contamination is considered a source of false-positive results. Under-sequenced aliquots are those with sequencing coverage (number of reads) significantly lower than the average sequencing coverage of other aliquots. Contamination between aliquots can occur in sequencing instruments. Aliquots with a coverage of the target clonal marker that is significantly lower compared to the average coverage of the same marker in other aliquots are considered as contaminated aliquotas. Z-score is used as a statistical metric for the detection of both under-sequenced aliquots and possible contaminations. After the exclusion of under-sequenced aliquots from analysis and contamination removal, aliquots with at least one sequencing read corresponding to the target clonal marker are considered positive aliquots, other aliquots are considered negative aliquots. Each aliquot can be positive for one target marker and negative for another. In the present example, 4 out of 32 aliquots were considered to be under-sequenced. After filtering, fifteen 300 ng aliquots, seven 30 ng aliquots, and six 3 ng aliquots were taken to the downstream analysis.

MRD presence is determined when at least one positive aliquot at least for one marker is detected. MRD levels (concentration of leukemic cells) are calculated using each target clonal marker separately. Among equal aliquotes, MRD is the number of positive aliquots (proxy to the number of leukemic cells) divided by the number of analyzed cells (one cell contains ˜6.5 pg DNA) as a first approximation. More precisely the number of leukemic cells can be calculated using the Poisson statistics model: N=−ln(1−n/m), where “N” is the number of leukemic cells in the sample (number of target molecules in the DNA sample), “n” is the number of positive aliquots, “m”-number of analyzed aliquots. When all aliquots are positive, a set of diluted aliquots is used to detect MRD levels. In the given example all fifteen 300 ng aliquots and all seven diluted 30 ng aliquots are positive for all target markers. Among the six remaining 3 ng aliquots TRBV20-1/J2-2 marker is present in 6 aliquots, one IGKV1D-43/Kde marker is present in 4 aliquots, another IGKV1D-43/Kde marker is present in 3 aliquots, TRDV2/D3 marker is present in 5 aliquots, IGKV1-39/J1 marker is present 4 aliquots, TRGV3/J1 is present in 5 aliquots. The number of positive aliquots allows to conclude that MRD levels (concentration of leukemic cells) calculated using separate MRD markers are >0.5%, 0.14%, 0.1%, 0.18%, 0.14%, 0.18%. Integration of these results across all 6 MRD markers allows to conclude that consensus MRD (max value across the markers) is present at the minimal frequency 0.5%.

TABLE 19
MRD level calculation for clinical samples.

| | | | | | | | | | Total | | | |
|---|---|---|---|---|---|---|---|---|---|---|---|---|
| | | Reads per target clonal markers | | | | | | Total | reads per | | | |
| DNA | | TRB | IGK | IGK | | IGK | | reads | target | | | |
| per | | V20- | V1D- | V1D- | TRD | V1- | TRG | per | V/D/J | Number of | Reads per | |
| aliquot | | 1J2-2 | 43Kde | 43Kde | V2D3 | 39J1 | V3J1 | aliquot | combinations | clonotypes | clonotype | Comments |
| 300 | ng | 863 | 0 | 1 | 253 | 10 | 84 | 20523 | 7485 | 2902 | 2.6 | |
| 300 | ng | 1158 | 3 | 1 | 325 | 10 | 110 | 28067 | 10299 | 3460 | 3.0 | |
| 300 | ng | 1273 | 0 | 3 | 318 | 25 | 120 | 33318 | 11983 | 3785 | 3.2 | |
| 300 | ng | 1498 | 3 | 8 | 501 | 21 | 179 | 49638 | 17153 | 4861 | 3.5 | |
| 300 | ng | 1683 | 7 | 10 | 701 | 27 | 240 | 55406 | 20025 | 5065 | 4.0 | |
| 300 | ng | 1645 | 4 | 12 | 689 | 21 | 219 | 53874 | 19810 | 4866 | 4.1 | |
| 300 | ng | 1948 | 7 | 2 | 674 | 33 | 255 | 63000 | 22954 | 5478 | 4.2 | |
| 300 | ng | 2013 | 5 | 5 | 720 | 35 | 197 | 58864 | 19760 | 4809 | 4.1 | |
| 300 | ng | 1004 | 2 | 5 | 293 | 16 | 134 | 26874 | 9767 | 3421 | 2.9 | |
| 300 | ng | 1085 | 3 | 2 | 345 | 17 | 88 | 31214 | 10770 | 3602 | 3.0 | |
| 300 | ng | 1147 | 5 | 6 | 280 | 16 | 125 | 36773 | 12595 | 3886 | 3.2 | |
| 300 | ng | 1275 | 7 | 7 | 367 | 16 | 164 | 40346 | 13794 | 4066 | 3.4 | |
| 300 | ng | 1156 | 1 | 3 | 408 | 11 | 139 | 31711 | 12141 | 3875 | 3.1 | |
| 300 | ng | 3 | 0 | 0 | 3 | 1 | 3 | 306 | 103 | 95 | 1.1 | under- |
| | | | | | | | | | | | | sequenced |
| 300 | ng | 1742 | 9 | 11 | 721 | 16 | 181 | 56466 | 19592 | 4876 | 4.0 | |
| 300 | ng | 1663 | 4 | 6 | 598 | 29 | 221 | 58471 | 18791 | 4735 | 4.0 | |
| MRD | | >0.0043 | 0.0019 | >0.004 | >0.0043 | >0.0043 | >0.0043 | | | | | |
| level, % | | | | 3 | | | | | | | | |
| 30 | ng | 412 | 5 | 2 | 98 | 6 | 136 | 13051 | 5451 | 875 | 6.2 | |
| 30 | ng | 280 | 1 | 4 | 196 | 12 | 84 | 13787 | 4951 | 843 | 5.9 | |
| 30 | ng | 5 | 0 | 0 | 3 | 0 | 1 | 195 | 66 | 60 | 1.1 | under- |
| | | | | | | | | | | | | sequenced |
| 30 | ng | 505 | 2 | 4 | 340 | 8 | 131 | 21644 | 9495 | 1006 | 9.4 | |
| 30 | ng | 783 | 3 | 5 | 179 | 24 | 127 | 30185 | 11336 | 1115 | 10.2 | |
| 30 | ng | 872 | 3 | 8 | 398 | 24 | 125 | 28240 | 11554 | 1178 | 9.8 | |
| 30 | ng | 861 | 4 | 6 | 456 | 31 | 179 | 32840 | 11705 | 1197 | 9.8 | |
| 30 | ng | 866 | 7 | 10 | 556 | 13 | 144 | 29342 | 11889 | 1141 | 10.4 | |
| MRD | | >0.022 | >0.022 | >0.022 | >0.022 | >0.022 | >0.022 | | | | | |
| level, % | | | | | | | | | | | | |
| 3 | ng | 3 | 0 | 0 | 9 | 0 | 13 | 2725 | 724 | 102 | 7.1 | |
| 3 | ng | 90 | 0 | 0 | 125 | 4 | 24 | 3114 | 1333 | 128 | 10.4 | |
| 3 | ng | 256 | 1 | 2 | 0 | 2 | 31 | 3807 | 1881 | 149 | 12.6 | |
| 3 | ng | 72 | 2 | 1 | 64 | 2 | 6 | 5780 | 2007 | 172 | 11.7 | |
| 3 | ng | 48 | 1 | 0 | 53 | 13 | 11 | 7807 | 2554 | 158 | 16.2 | |
| 3 | ng | 1 | 0 | 0 | 1 | 0 | 0 | 112 | 38 | 38 | 1.0 | under- |
| | | | | | | | | | | | | sequenced |
| 3 | ng | 0 | 0 | 0 | 2 | 0 | 1 | 229 | 74 | 68 | 1.1 | under- |
| | | | | | | | | | | | | sequenced |
| 3 | ng | 2 | 3 | 5 | 160 | 0 | 0 | 9022 | 2815 | 186 | 15.1 | |

| MRD | >0.5 | 0.14 | 0.1 | 0.18 | 0.14 | 0.18 |
|---|---|---|---|---|---|---|
| level, % | | | | | | |
| Consensus | >0.5% | | | | | |
| MRD | | | | | | |
| level | | | | | | |

### Example 7. Quantitative Bias Correction via Weighted Versus Unweighted Non-Functional Rearrangement Frequencies Analysis

This example demonstrates how gene segment frequency correction in the VDJ rearrangement data studied in Example 6 can be performed based on the relative abundance of non-functional (e.g., non-productive or out-of-frame) clonotypes. Non-productive (containing the stop codon) or out-of-frame TCR/BCR rearrangements cannot form a functional receptor, and therefore they are not subjected to antigen-specific selection and clonal expansion (Murugan et al., 2012, PNAS 109:16161-16166).

Therefore, such rearrangements can be used as passenger genomic variations, with clonal frequencies that change randomly, following the frequency changes of the second functional (in-frame, no stop codon) TCR/BCR allele present in the same T/B cell clone. The formation of in-frame and out-of-frame allele combinations in the same cell is also a stochastic and independent process in terms of V- and J-gene frequency.

Therefore, reproducible deviation of out-of-frame V- and J-gene frequencies from the initial recombinational frequencies observed in the sequenced repertoire dataset (for the same multiplex PCR primer set, same production lot, etc) can be determined as a result of artificial aberration caused by PCR amplification rather than immune repertoire evolution.

In this example, out-of-frame clonotypes were used as a natural calibrator to measure amplification bias. This allowed for changing the primer concentration in order to reduce such bias, and finally quantitatively correct the remaining amplification bias in immune repertoire data.

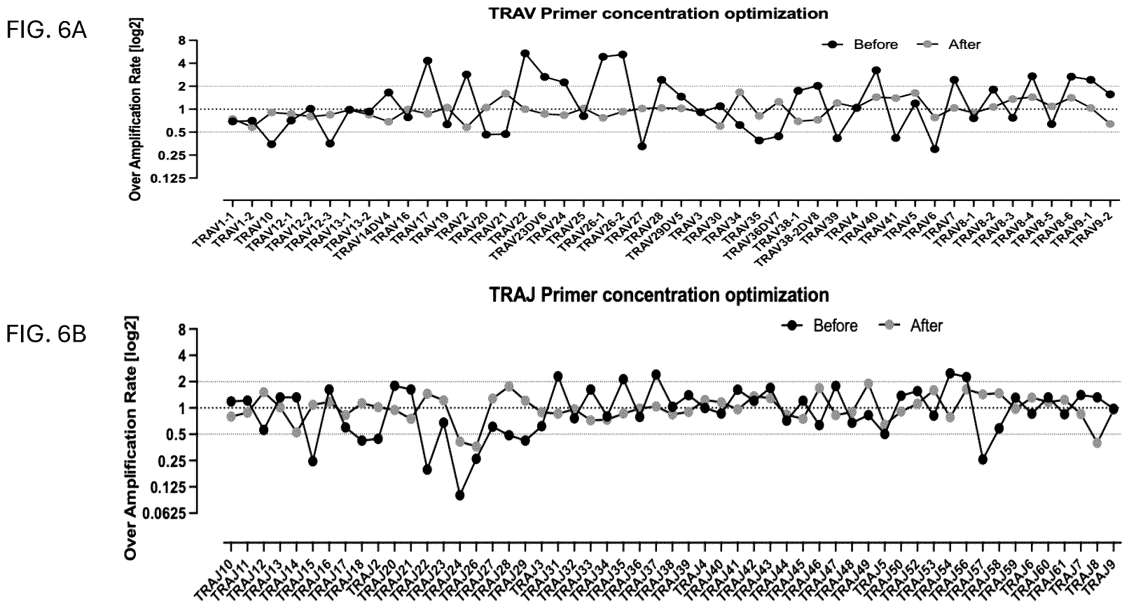

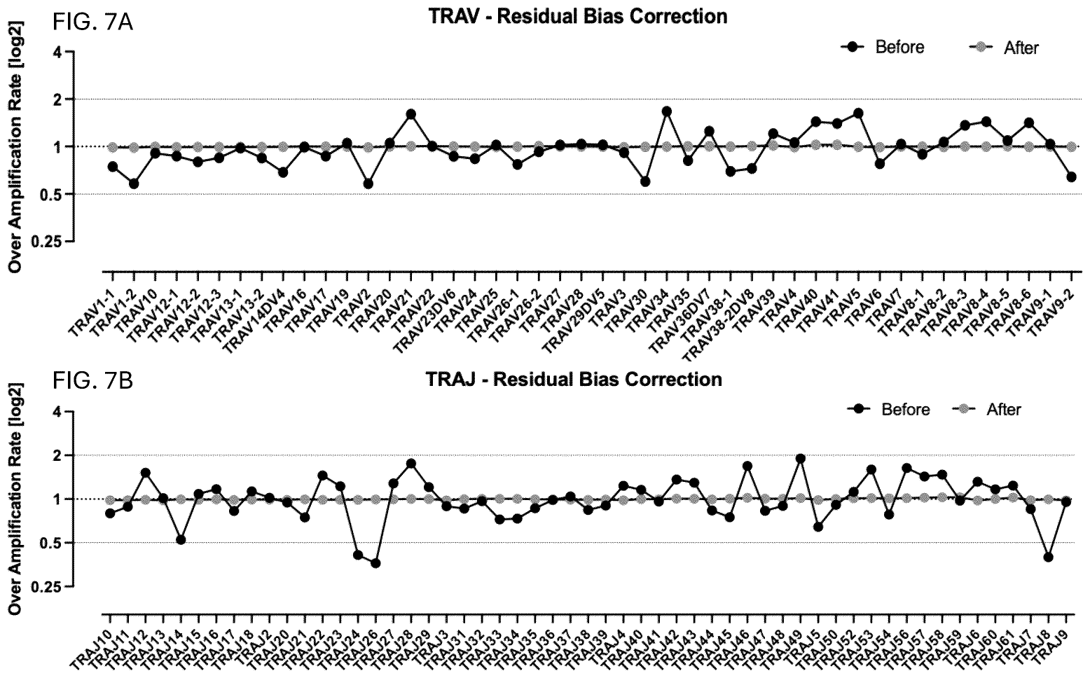

Changes in TRAV and TRAJ overamplification rate during primer concentration optimization are plotted in FIGS. 6A and 6B. Changes in TRAV and TRAJ overamplification rate after final computational bias correction are plotted in FIGS. 7A and 7B. Overamplification rates (OAR) are calculated according to the formula:

$$OAR ( Vi ) = \frac{RC ( Vi ) / {\sum }_{1}^{N} RC ( Vi )}{UCN ( Vi ) / {\sum }_{1}^{N} UCN ( Vi )}$$

$$OAR ( Ji ) = \frac{RC ( Ji ) / {\sum }_{1}^{N} RC ( Ji )}{UCN ( Ji ) / {\sum }_{1}^{N} UCN ( Ji )}$$

where OAR—OverAmplification Rate, RC—read counts, UCN—unique out-of-frame clones, Vi-particular V gene, Ji-particular J gene.

The correction coefficient for computational bias removal is calculated by simply multiplying OAR (Vi) by OAR (Ji). To correct amplification bias, each clonotype should be divided by the corresponding correction coefficient.

As can be seen in both FIGS. 6 and 7, the methods were effective in overcoming amplification bias at the stage of wet-lab adaptome library preparation and computational analysis of the obtained adaptome after high-throughput sequencing.

What is claimed is:

1. A method of determining the presence and quantification of minimal residual disease (MRD) in patients with hematological malignancies, the method comprising:
a. detecting rearrangements of T cell receptor genes and B cell receptor genes characteristic for malignant clones in an initial sample from a patient using:
a multiplex polymerase chain reaction (PCR) with isolated genomic DNA wherein the PCR reaction comprises a library of oligonucleotides amplifying one or more of the following: TRα/β/γ/δ, IgH/K/λ, DJ, DD, VD, and kappa-deleting element (KDE) rearrangements in a single multiplex mixture of oligonucleotides;
high-throughput sequencing of the obtained PCR products;
extracting complete and incomplete rearrangements by mapping potential rearrangements to a library comprising V, D, and J gene segments, kappa deletion element (KDE), and IGKC intron sequences, wherein the extraction is performed using a semi-global alignment algorithm to identify flanking sequences, followed by a clustering algorithm correcting for PCR and sequencing errors to assemble complete and/or incomplete rearrangement clonotypes;
determining precise clonotype sizes from hematological malignancy-related repertoire; and
malignancy-related clonotype detection;
b. performing follow-up monitoring of MRD in at least 4 independent PCR reactions with isolated and quantified genomic DNA obtained at a follow-up time point; wherein the concentration of a malignant clone in the follow-up time point is determined based on the proportion of independent PCR reactions where the corresponding rearrangement characteristic for the malignant clone is observed.

2. The method of claim 1, wherein the library of oligonucleotides for multiplex PCR is designed to decrease primer dimer formation and minimize nucleotide diversity.

3. The method of claim 1, wherein the library of oligonucleotides is designed by a method comprising:
a. dividing target genomic DNA regions into k-mers of a certain length;
b. clustering the target genomic DNA regions using Hamming distances not exceeding 4, not exceeding 5, or not exceeding 6, or not exceeding 7 nucleotides; and
c. identifying clusters of highly similar k-mers according to the selected Hamming distance.

4. The method of claim 3, wherein the k-mers have a length of at least 16 bases.

5. The method of claim 3, wherein from the clusters identified, tables of 5-mers in the reverse orientation and 5-mers at the end of 3′ end of the nucleotide sequences in the forward orientation are made.

6. The method of claim 5, wherein the 5-mers tables are intersected pairwise, and the primer set with the lowest 5-mers overlap is selected for further analysis.

7. The method of claim 4, wherein, to equilibrate the annealing temperature of the primers, up to 14 additional target-site-related nucleotides are added to the 5′-end of 18-mers.

8. The method of claim 5, wherein the intersection procedure using 5-mers is repeated.

9. The method of claim 3, wherein at least 2 working sets of forward primers and at least 2 working sets of reverse primers are generated.

10. The method of claim 9, wherein the reverse primer set comprises primers for J-genes, downstream introns for D-genes and KDE.

11. The method of claim 9, wherein the forward primer set comprises primers for V-genes, upstream introns for D-genes and C-intron for IgK.

12. The method of claim 9, wherein the multiplex primer sets generated can be used for separate amplification of complete VJ and/or VDJ rearrangements at TRα, TRβ, TRγ, TRδ, IgH, IgK, IgL loci; partial DJ rearrangements at TRβ, TRδ, IgH loci; VD and DD rearrangements at TRδ and TRβ loci; chimeric TRDV-TRAJ rearrangements; or Kappa deletion rearrangements.

13. The method of claim 12, wherein dual indexing of each aliquot amplicon and introduction of adapters for sequencing are performed using “step-out” PCR.

14. The method of claim 12, wherein the average lengths of target amplicons in the 4 multiplex primer sets are: set 1—about 120 bp, set 2—about 160 bp, set 3—about 240 bp, and set 4—about 280 bp.

15. The method of claim 9, wherein amplicons obtained with a set of primers cannot be a matrix for PCR with the subsequent set of primers.

16. The method of claim 1, wherein the concentrations of primers in the multiplex mixture are optimized by analyzing the frequency ratios of non-functional rearrangements of T- and B-cell receptor genes.

17. The method of claim 16, wherein an overamplification rates (OAR) is determined for at least one rearrangement, and the concentration of the primer detecting the rearrangement is adjusted to correct for the amplification bias including overamplification and underamplification).

18. The method of claim 1, wherein an overamplification rates (OAR) is determined for at least one rearrangement to computationally correct for the amplification bias including overamplification and underamplification.

19. The method of claim 1, wherein a ratio of weighted frequencies (proportion of reads) to unweighted frequencies (proportion of clonotypes) for V, D and J genes is determined.

20. The method of claim 19, wherein, if the ratio is a substantial deviation from 1, it is indicative of quantitative bias occurring during amplification.

21. The method of claim 20, wherein the concentrations of primers in the multiplex mixture is selected to minimize the deviation from the expected value of one for the ratio of weighted frequencies to unweighted frequencies.

22. The method of claim 1, wherein the MRD detection sensitivity depends exclusively on the available input DNA and sequencing coverage.

23. The method of claim 1, wherein TRα/β/γ/δ, IgH/K/λ, DJ, DD, VD, and kappa-deleting element (KDE) rearrangements are detected in a single multiplex mixture of oligonucleotides.
